(19) United States
(12) Patent Application Publication
(10) Pub. No.: US 2017/0003275 A1
(43) Pub. Date: Jan. 5, 2017

(54) METHOD TO IDENTIFY COMPOUNDS ABLE TO BIND TO THE ROSSMANN FOLD OF C-TERMINAL-BINDING PROTEINS, IDENTIFIED COMPOUNDS AND MEDICAL USES THEREOF

(71) Applicant: CONSIGLIO NAZIONALE DELLE RICERCHE, Rome (IT)

(72) Inventors: Antonino COLANZI, Rome (IT); Alberto LUINI, Rome (IT); Daniela CORDA, Rome (IT)

(21) Appl. No.: 14/891,565

(22) PCT Filed: May 16, 2014
(86) PCT No.: PCT/EP2014/060143

§ 371 Date: Nov. 16, 2015

(30) Foreign Application Priority Data
May 16, 2013 (IT) RM2013A000295

(51) Int. Cl. G01N 33/50

(52) U.S. Cl. CPC G01N 33/5011; G01N 2500/10; G01N 2500/04

(57) ABSTRACT

The invention refers to a method for identifying an anti-tumoral and/or anti-proliferative and/or an inhibitor of the fission machinery involved in mitotic Golgi partitioning and/or a molecule modulator of CtBP corepressor activity by testing their affinity binding for the Rossmann fold of C-terminal-binding proteins (CtBPs); to said identified molecules and to the use thereof as anti-proliferative and/or anti tumoral agents.

### FIELD OF THE INVENTION

The present invention refers to a method to identify compounds able to bind to the Rossmann Fold of C-terminal-binding proteins, identified compounds and medical uses thereof, in particular as pro-apoptotic agents and anti tumorals.

### BACKGROUND OF THE INVENTION

The modification of proteins by mono-ADP-ribosylation involves the transfer of a single ADP-ribose (ADPR) from NAD⁺ to specific aminoacids in target proteins by mono-ADP-ribosyltransferases (1-3). The mono-ADP-ribosylation reaction was characterized first in bacteria, where ADP-ribosyltransferases have roles as toxins (e.g., cholera and pertussis toxins (1)). More recently, this reaction has been characterized also in eukaryotic cells, where a large group of mono-ADP-ribosyltransferases has been identified and proposed to be involved in the regulation of numerous physiological functions (4). Authors have previously reported that the fungal toxin brefeldin A (BFA), which is a macrocyclic lactone that is widely used in studies of membrane trafficking (see below), induces the ADP-ribosylation of the C-terminal-binding protein-1 short form/BFA-ADP-ribosylation substrate (CtBP1-S/BARS; for brevity BARS) with high affinity and selectivity, and of the glycolytic enzyme GAPDH with much lower efficiency (5, 6). Here, authors explore the molecular mechanisms and the possible function of the BFA-dependent ADP-ribosylation of BARS. BARS is structurally related to the D2-hydroxy acid dehydrogenase family and is a member of the C-terminal-binding proteins (CtBPs) family, that includes five proteins, that have been implicated in both fission of intracellular membranes and transcriptional repression: CtBP1-L (NCBI Accession number: U37408.1), CtBP1-S/BARS (BARS) (NCBI Accession numbers: protein: Q9Z2F5.3, nucleotide: AF067795.2), CtBP2-L (NCBI Accession numbers: protein: AAC39603.1, nucleotide: AF016507.1), CtBP2-S (SEQ ID NO: 22, corresponding to aa. 26-445 of the aa. sequence of NCBI Accession number AAC39603.1; see also: Verger A, Quinlan K G, Crofts L A, Spanò S, Corda D, Kable E P, Braet F, Crossley M. Mechanisms directing the nuclear localization of the CtBP family proteins. Mol Cell Biol. 2006 July; 26(13):4882-94. PubMed PMID: 16782877; PubMed Central PMCID: PMC1489157), and RIBEYE (NCBI Accession numbers: protein: AAG45951.1, nucleotide: AF222711.1) (7). The CtBPs are involved in two processes, one in the cell cytosol and the other in the nucleus (7). In the cytosol, BARS controls the membrane-fission machinery that drives the formation of post-Golgi carriers (8, 9), endocytic fluid-phase carriers (8, 10), COP1-coated vesicles (11), and the partitioning of Golgi during the G2 phase of the cell cycle, a step that also controls cell entry into mitosis (12, 13). In the nucleus, members of the CtBP protein family act as transcription co-repressors, and thus regulate numerous cellular functions, including epithelial differentiation, tumorigenesis and apoptosis (14, 15). Whether the nuclear and cytoplasmic functions of BARS are related remains unclear to date. CtBPs consists of two compact domains separated by a deep cleft, named also as the Rossmann fold, which is a structural motif present in proteins that bind nucleotides, in particular the cofactor NAD+/NADH. The NAD binding domain is present as Rossmann fold in many dehydrogenases. The Rossmann fold is composed of two parallel groups of three beta strands, connected by alpha helices (organized in the order beta-alpha-beta-alpha-beta) and characterized by: —a phosphate binding consensus sequence GXGXXG (SEQ ID NO: 1) wherein X is any of the 20 natural amino acids, —a basic residue (Arg or Lys) at the beginning of the first beta strand, —an acid residue (Glu or Asp) at the end of the second beta strand, and —at least six hydrophobic residues (16, 17). Notably, NADH stimulates the dimerization of CtBPs and the recruitment of their binding partners, enhancing the corepressor activity. In fact, mutations of the NADH-interaction domain of CtBPs interfere with its ability to act as transcriptional corepressors (Zhang et al., 2002). Significantly, the Rossmann fold of CtBP1 and CtBP2 is conserved. BFA is a toxin produced by several fungi (e.g., Eupenicillium brefeldianum, Alternaria carthami), whose role in nature is not well understood. It has been shown to induce necrosis of the leaf tissue in safflower (leaf spot diseases), probably to facilitate colonization by the fungi (18). As a research tool, BFA has been characterized extensively and used to analyze the mechanisms of membrane transport. Its best-known effect is the induction of the formation of numerous long tubules from the Golgi complex, which then fuse with the endoplasmic reticulum (ER), thereby mediating the redistribution of resident Golgi proteins into the ER (19), and hence causing a rapid and reversible block of secretion (19). At the molecular level, this effect of BFA is mediated by the inhibition of the GTPase exchange factor acting on the small Ras-like GTPase ARF, and by the release of ARF from the Golgi complex along with a set of proteins that are regulated by ARF (20). Whether the effects of BFA on Golgi tubulation and disassembly are linked to those of the ADP-ribosylation of BARS remains unclear (7). It has been proposed that ADP-ribosylation of BARS contributes to the disassembly of the Golgi complex, at least under certain conditions (21), by inhibiting the fission of Golgi tubules (7, 22).

### DESCRIPTION OF THE INVENTION

Authors show that ADP-ribosylation of CtBP1-SEARS by BFA occurs via a non-conventional mechanism that comprises two steps: (i) synthesis of a BFA-ADP-ribose conjugate (BAC) by the ADP-ribosyl cyclase CD38; and (ii) covalent binding of the BFA-ADP-ribose conjugate into the CtBP1-SEARS NAD⁺-binding pocket domain (Rossmann fold). Modeling studies suggested that the ADP ribose portion of BAC is involved in recognition and binding to the BARS nucleotide-binding cleft, while the BFA portion is involved in covalent binding to His304 of CtBPs. This results in the locking of CtBP1-S/BARS in a dimeric conformation, which prevents its binding to interactors known to be involved in membrane fission, and hence in the inhibition of the fission machinery involved in mitotic Golgi partitioning. As this inhibition can lead to arrest of the cell cycle in G2, these findings provide a strategy for the design of pharmacological blockers of the cell cycle in tumor cells that express high levels of CD38. To determine whether CtBPs loss-of-function can provide an antitumour strategy, authors depleted CtBP1 and CtBP2 (both short and long splice variants (7)) by transfection siRNAs in HeLa cells (a cell line from human cervix adenocarcinoma). This knock-down (KD) induced a strong reduction of cell growth and increased cell sensitivity to apoptotic stimuli and chemotherapy agents. To identify genes that are regulated by CtBPs in Hela cells and that could be responsible of the effects on apoptosis and proliferation, authors first used qRT-PCR and Western blotting and found that the proapoptotic gene BIK (NCBI Accession numbers: protein: CAG30276.1, nucleotide: U34584.1) was consistently and highly upregulated following depletion of CtBPs. To extend this investigation, a microarray analysis has also been performed comparing HeLa cells depleted of CtBPs with control cells transfected with non-targeting siRNA. This investigation has led to the identification of several genes that are upregulated in KD cells and that are involved in regulation of cell proliferation, cell death and response to stress. Therefore, the CtBPs are attractive cancer drug targets since they encode a druggable binding domain, the “Rossmann fold”, involved in the regulation of corepressor function. Authors' previous work has shown that BARS is able to bind NAD(H) and Acyl-CoAs at the same site (28). This different binding regulates BARS conformation: the protein can shift between a monomeric and dimeric state, determined by Acyl-CoA and NAD(H) binding, respectively. These two conformations are crucial for function as they facilitate the transcriptional activity (dimer) or fission regulation (monomer) of the CtBP proteins (7). Thus, the identification of BAC and the definition of the molecular mechanism of its binding to BARS has offered the possibility of setting up an assay to select molecules that can bind the Rossmann fold by testing their capability of inhibiting BAC binding to recombinant BARS. Through this assay, authors have found that several molecules, as e.g. dicumarol, coumermycin A1 and gossypol, bind to the Rossmann fold of BARS with high affinity. More importantly, these molecules increase the expression of the CtBP target gene BIK and are able to induce apoptosis and reduce cell growth, thus mimicking the effects of CtBPs-KD. Thus, present data indicate that the Rossmann fold of CtBPs can be exploited for the design of antitumoral molecules that specifically bind this structural domain, thus competing with NADH and/or altering their conformation, thus affecting either the membrane fission- or the transcription-related functions of CtBP/BARS. These molecules are expected to inhibit mitotic entry and tumour progression in specific types of cancer.

Object of the invention is a method for identifying a molecule acting as an anti-tumoral and/or an anti-proliferative and/or an inhibitor of the fission machinery involved in mitotic Golgi partitioning and/or a modulator of C-terminal-binding proteins (CtBPs) corepressor activity, comprising the steps of:
assaying candidate molecules for their affinity binding for the Rossmann fold of C-terminal-binding proteins (CtBPs);
selecting molecules having an high affinity binding for the Rossmann fold of C-terminal-binding proteins (CtBPs);
testing such high affinity binding molecules for their capacity of inhibiting proliferation and/or inducing an apoptotic response in a cell system.

Said candidate molecules are preferably previously selected from database through virtual docking on the C-terminal-binding proteins (CtBPs), preferably on the CtBP1-SEARS (BARS) protein (BARS GenBank accession No. AF067795.2).

Said Rossmann fold preferably belongs to the CtBP1-SEARS (BARS) protein (BARS GenBank accession No. AF067795.2).

Proteins encoded by ortologhs of the CtBP1-S/BARS (BARS) gene econding for the above BARS protein, for e.g. CtBP1-SEARS (BARS) protein of human origin, are comprised within the definition of CtBP1-S/BARS (BARS) protein.

The tumor is preferably a solid tumor, more preferably the solid tumor is breast, colon, lung cancer or melanoma.

Preferably, the cell system used for testing expresses high levels of CD38.

Another object of the invention is a molecule obtainable by the above disclosed method for use as anti tumoral and/or anti proliferative agent wherein the molecule is selected from the group consisting of:

a) a BFA-ADPR conjugate, said conjugate being formed by ADP-ribosyl cyclase activity, or
b) an inhibitor of binding to BARS of the BFA-ADPR conjugate and/or of NAD/NADH and/or of Acyl CoAs, with the proviso that said inhibitor is not gossypol.

The tumor is preferably characterized by high levels of CD38 expression.

In a preferred aspect, the inhibitor is selected from the group consisting of:

the compound of formula (I):

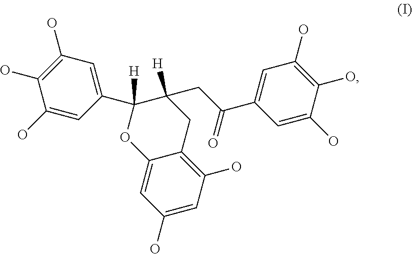

the compound of formula (II):

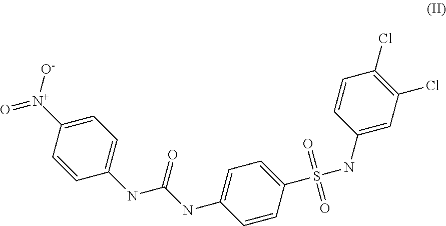 [SMILES: O=C(Nc1ccc([N+](=O)[O-])cc1)Nc1ccc(S(=O)(=O)Nc2ccc(Cl)c(Cl)c2)cc1]

dicumarol, coumermycin A1, salts and derivatives thereof.

A further object of the invention is a molecule able to selectively and with high affinity bind to the Rossmann fold of C-terminal-binding proteins (CtBPs) for use as anti tumoral and/or anti proliferative agent wherein the molecule is selected from the group consisting of:

a) a BFA-ADPR conjugate, said conjugate being formed by ADP-ribosyl cyclase activity, or
b) an inhibitor of binding to BARS of the BFA-ADPR conjugate and/or of NAD/NADH and/or of Acyl CoAs, with the proviso that said inhibitor is not gossypol.

Preferably, the tumor is characterized by high levels of CD38 expression.

In a preferred embodiment, the inhibitor is selected from the group consisting of:

the compound of formula (I):

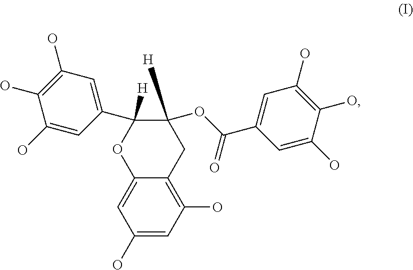

the compound of formula (II):

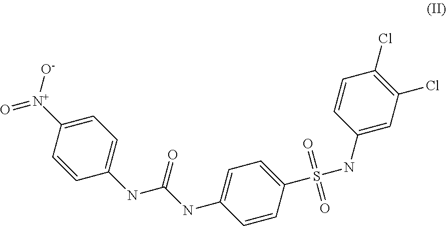 [SMILES: O=C(Nc1ccc([N+](=O)[O-])cc1)Nc1ccc(S(=O)(=O)Nc2ccc(Cl)c(Cl)c2)cc1]

dicumarol, coumermycin A1 salts and derivatives thereof.

The tumor is preferably a solid tumor, said solid tumor is more preferably breast, colon, lung cancer or melanoma.

Another object of the invention is a method of treatment of a tumor, comprising administering to a subject in need thereof an effective amount of a molecule obtainable by the above method, wherein the molecule is selected from the group consisting of:

a) a BFA-ADPR conjugate, said conjugate being formed by ADP-ribosyl cyclase activity, or
b) an inhibitor of binding to BARS of the BFA-ADPR conjugate and/or of NAD/NADH and/or of Acyl CoAs, with the proviso that said inhibitor is not gossypol.

A further object of the invention is a method of treatment of a tumor, comprising administering to a subject in need thereof an effective amount of a molecule able to selectively and with high affinity bind to the Rossmann fold of C-terminal-binding proteins (CtBPs), wherein the molecule is selected from the group consisting of:

a) a BFA-ADPR conjugate, said conjugate being formed by ADP-ribosyl cyclase activity, or
b) an inhibitor of binding to BARS of the BFA-ADPR conjugate and/or of NAD/NADH and/or of Acyl CoAs, with the proviso that said inhibitor is not gossypol.

The terms “treat” or “treatment” as well as words stemming therefrom, as used herein, do not necessarily imply 100% or complete treatment. According to the present invention, an “effective amount” of a composition is one which is sufficient to achieve a desired biological effect, in this case e.g. a decrease in the mass tumour or a decrease in metastatic potential. It is understood that the effective dosage will be dependent upon the age, sex, health, and weight of the recipient, kind of concurrent treatment, if any, frequency of treatment, and the nature of the effect desired. The preferred dosage can be tailored to the individual subject, as is understood and determinable by one of skill in the art, without undue experimentation. Examples of ranges of effective doses of the above molecules of the invention (from 1 mg/kg to 100 mg/kg, in particular systemically, topically, locally and orally administered) are not intended to limit the invention and represent preferred dose ranges. The disclosed molecules can be administered in a composition (e.g., pharmaceutical composition) that can comprise at least one excipient (e.g., a pharmaceutically acceptable excipient), as well as other therapeutic agents (e.g., anti-cancer agents). The composition can be administered by any suitable route, including parenteral, topical, oral, or local administration. The pharmaceutically acceptable excipient is preferably one that is chemically inert to the molecules above disclosed and one that has little or no side effects or toxicity under the conditions of use. Such pharmaceutically acceptable carriers include, but are not limited to, water, saline, Cremophor EL (Sigma Chemical Co., St. Louis, Mo.), propylene glycol, polyethylene glycol, alcohol, and combinations thereof. The choice of carrier will be determined in part by the particular compound as well as by the particular method used to administer the composition. Accordingly, there is a wide variety of suitable formulations of the composition. The pharmaceutical composition in the context of an embodiment of the invention can be, for example, in the form of a pill, capsule, or tablet, each containing a predetermined amount of one or more of the above molecules and preferably coated for ease of swallowing, in the form of a powder or granules, or in the form of a solution or suspension. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986). The concentration of a molecule of the invention in the pharmaceutical formulations can vary, e.g., from less than about 1%, usually at or at least about 10%, to as much as 20% to 50% or more by weight, and can be selected primarily by fluid volumes, and viscosities, in accordance with the particular mode of administration selected. Methods for preparing administrable (e.g., parenterally administrable) compositions are known or apparent to those skilled in the art and are described in more detail in, for example, Remington's Pharmaceutical Science (17th ed., Mack Publishing Company, Easton, Pa., 1985). In addition to the aforedescribed pharmaceutical compositions, the above moelcules can be formulated as inclusion complexes, such as cyclodextrin inclusion complexes, or liposomes. Liposomes can serve to target the molecules to a particular tissue. Many methods are available for preparing liposomes, as described in, for example, Szoka et al., Ann. Rev. Biophys. Bioeng., 9:467 (1980) and U.S. Pat. Nos. 4,235,871, 4,501,728, 4,837,028, and 5,019,369.

The compound of formula (I) is also herein defined as compound 7, C7 or (−)-Epigallocatechin gallate.

The compound of formula (II) is also herein defined as compound 11, C11 or N-(3,4-dichlorophenyl)-4-{[(4-nitrophenyl)carbamoyl]amino}benzenesulfonamide.

In the present invention, the term “CtBPs” or “C-terminal-binding proteins” includes BARS and all the CtBP proteins or isoforms, which are members of the CTBP protein family, and proteins coded by orthologhs and homologs of the genes encoding for BARS and said CtBP proteins or isoforms.

The term “derivative” as used herein means a chemically modified molecule or an analogue thereof, wherein at least one substituent is not present in the unmodified molecule or an analogue thereof, i.e. a peptide which has been covalently modified. Typical modifications are amides, carbohydrates, alkyl groups, acyl groups, esters and the like.

### FIGURE LEGENDS

The present invention will be described by means of non limiting examples referring to the following figures:

FIG. 1. ADP-ribosylation of BARS is mediated by formation of a conjugate between BFA and ADPR. (A) One-step reaction: total rat-brain membrane fractions (ME) and rat cytosol were incubated for 1 h at 37° C. with 30 μM total NAD (spiked with 5 μCi [³²P]-NAD⁺), in the absence and presence of BFA (80 μg/ml). Two-step reaction: total rat-brain membrane fractions, 30 μM total NAD (spiked with 5 μCi [³²P]-NAD⁺) and BFA (80 μg/ml) were combined as indicated, and incubated for 1 h at 37° C.; the membranes were then removed by centrifugation. The resulting supernatant was ultrafiltered (Microcon YM-3) and incubated with rat cytosol, for 1 h at 37° C. The samples were analyzed by SDS-PAGE and autoradiography. (B) Rat-brain membranes, [³²P]-NAD⁺ and BFA were incubated as indicated in the Methods. Membranes were removed by centrifugation and the supernatants were ultrafiltered. The resulting filtrate was loaded onto an HPLC C18 reverse-phase column and eluted with a nonlinear gradient of phosphate buffer containing tetrabutyl ammonium chloride (1^{st} column). The fractions that induced ADP-ribosylation were eluted at 100% non linear gradient of buffer B, and were recovered, lyophilized, and loaded onto an HPLC C18 RP column for the second purification step in the absence of tetrabutyl ammonium chloride. The metabolite of interest eluted at 50% buffer B after 17 min of the gradient (2^{nd} column). (C) The purified metabolite was incubated with rat-brain cytosol, separated by SDS-PAGE and analyzed by autoradiography. (D) Molecular structure of BFA. (E) Effect of BFA analogs on ADP-ribosylation of GADPH and BARS. (F) Proposed structure of BAC.

FIG. 2. Modeling of BAC binding to BARS View of the BARS nucleotide binding site modeled with BAC molecule. Residues relevant for the BAC interaction and for catalysis are shown. Hydrogen bonds are indicated as arrows with dashed lines.

FIG. 3. CD38 can support BAC synthesis in intact cells (A) Total membrane fractions from control (CD38 (−)) and CD38 (+) HeLa cells were incubated for 2 h at 37° C. with recombinant His-BARS and 30 μM total NAD (spiked with 5 μCi [³²P]-NAD⁺), in the absence and presence of BFA (80 μg/ml). The samples were analyzed by SDS-PAGE and autoradiography (AR [³²P]); total BARS levels were analyzed by Western blotting. (B) CD38(+) HeLa cells were transfected with wild-type YFP-BARS (BARS WT) or YFP-BARS with the His304 point mutation (BARS H304A). After 24 h, the cells were treated with 80 μg/ml BFA for 4 h at 37° C., in the presence of 5 mM extracellular NAD⁺. YFP-BARS was immunoprecipitated using an anti-BARS antibody and the modified protein was revealed using an anti-BFA-specific antibody. Total BARS levels are shown in the bottom panel. (C) CD38 (+) HeLa cells were transfected with YFP-BARS. After 24 h, the cells were treated with 80 μg/ml BFA for 1 h or 4 h at 37° C., in the presence or absence of exogenously added 5 mM NAD⁺, as indicated. YFP-BARS was immunoprecipitated from total lysates using an anti-BARS antibody, and the modified protein was revealed using an anti-BFA-specific antibody. Total levels of BARS are shown in the bottom panel.

FIG. 4. BAC affects the interactions of BARS with its partners involved in fission. (A) In-vitro GST pull-down assay. Equimolar amounts of GST-PAK1, GST-14-3-3γ, GST-E1A or GST were incubated with 5 μg His-BARS or His-BARS bound to BAC, in GST incubation buffer (20 mM Tris at pH 8.0, 100 mM KCl, 1 mM EDTA, 0.2% Triton X-100 and protease inhibitors) for 2 h at 4° C., with gentle agitation. After this step, 25 μl glutathione Sepharose 4B matrix (previously equilibrated in GST incubation buffer) was added to each of the samples, which were then further incubated for 1 h at 4° C. The suspensions were then centrifuged at 500×g for 5 min, to sediment the matrix. Following extensive washing of the beads, the interacting proteins were eluted with 100 mM Tris at pH 8.0, 20 mM reduced glutathione, 5 mM DTT. The samples were subjected to SDS-PAGE and immuno-blotted with antibodies directed against GST or BARS. (B) Gel filtration patterns of rat-brain cytosol treated as indicated. Five milligram of rat-brain cytosol were applied to a Superose 12 High Resolution 10/30 (Amersham Pharmacia) gel filtration column, as previously described (21). The eluted proteins were collected using an AKTA FPLC system (Amersham Pharmacia) and detected by monitoring absorbance at 280 nm. The collected fractions were separated by SDS-PAGE and immunoblotted with an anti-BARS antibody. (C) Golgi fragmentation assay. Digitonin-permeabilised NRK cells were incubated with mitotic cytosol pre-incubated with HPLC-purified BAC (BAC) or buffer alone. Golgi fragmentation was evaluated and quantified by immunofluorescence using an antibody against giantin (see (13)). (D) Quantification of the mitotic index of cells grown on coverslips and arrested in the G2 phase of cell cycle. HeLa cells were grown on fibronectin-coated glass coverslips. Once attached, they were incubated in growth medium plus 9 μM RO-3306 for 20 h, to accumulate the cells in G2 phase (23). The G2-block was removed by washing out RO-3306. One hour before the G2 block release, the cells were microinjected with BAC or buffer alone (Hepes 20 mM (pH 7.4), sucrose 10 mM), previously mixed with dextran-FITC as tracer of microinjection. The cells were fixed 40 minutes after the G2 block release and stained with the DNA dye Hoechst 33342. Quantification data are means±SD from three independent experiments, each carried out in duplicate. More than 200 cells were microinjected for each condition. (E) HeLa cells were synchronized for cell cycle transition using a double thymidine treatment (13). Briefly, cells were maintained in growth medium plus 2 mM thymidine for 16 h and then rinsed and maintained in growth medium for 8 h. The cells were then maintained in thymidine for an additional 16 h before the final release of the cell cycle arrest. Eight hours after the release, the cells were treated with epoxy-BFA (1 μg/ml) or vehicle alone and then fixed two hours later. DNA was labeled with Hoechst. The mitotic cells were estimated by fluorescence microscopy measuring the number of cells showing condensed chromosomes. Quantification data are means±SD from two independent experiments, each carried out in duplicate. More than 400 cells were counted for each condition.

FIG. 5. Knockdown of CtBP1/2 reduces cell growth and sensitize to apopotic stimuli. Hela cells were grown on coverlisps and transfected with 50 nM of non-targeting siRNAs (dark grey bar) or 50 nM of a single siRNA duplex to target both CtBP1 and CtBP2 (light grey bar). (A) At the indicated time point after transfection, the cells were trispinized and counted. (B) Following 48 h of transfection, the cells were either left untreated, or treated for 1 h with the indicated concentrations of staurosporin and etoposside to induce an apoptotic response. The samples were then analyzed by SDS-PAGE and western blot to monitor apoptotosis trough PARP1 cleavage (as described in (24)). Legend: black bar (non transfected); ON, overnight treatment).

FIG. 6. Identification of lead molecules that bind the Rossman fold. A) Recombinant BARS (2 μg) was first incubated in ADP-ribosylation buffer for 30 min at room temperature with 100 μM dicumarol and then for 1 h at 37° C. with the indicated concentrations of BAC in a total volume of 50 μl. The reactions were stopped by adding sample buffer followed by samples analysis by SDS-PAGE and Western blotting using an anti-BFA antibody to detect BAC binding. B) Recombinant BARS (2 μg) was first incubated in ADP-ribosylation buffer for 30 min at room temperature with 100 μM dicumarol, coumermycin A1 or Gossypol and then for 1 h at 37° C. with 2 μM of BAC in a total volume of 50 μl. The reactions were stopped by adding sample buffer followed by samples analysis by SDS-PAGE and Western blotting using an anti-BFA antibody to detect BAC binding. C) Quantitation of BAC binding to BARS revealed by western blots shown in (B). Proteins were quantified by densitometry using the ImageJ software.

FIG. 7. Identification of a CtBP-reporter gene. Hela cells were grown in 12 multiwell plates. The cells were not-treated (Mock) or transfected for 48 h with non-targeting (non-targeting) and CtBPs targeting siRNAs (CtBP1+CtBP2). The cells were then analysed by immunoblotting with antibodies against CtBP1, CtBP2 and GAPDH (as protein levels control) (A) and by qRT-PCR to monitor expression levels of a series of putative CtBP target genes derived from literature (EMT markers, regulators of cell proliferation and pro-apoptotic genes, such as p21 (NCBI Accession numbers: protein: AAB29246.1, nucleotide: S67388.1), Bax (NCBI Accession numbers: protein: AAB35593.1, nucleotide: L22473.1), Noxa (NCBI Accession numbers: protein: AAH32663.1, nucleotide: BC032663.1), Bik and PERP (NCBI Accession numbers: protein: AAH10163.1, nucleotide: NM_022121.4) (B) and a microarray analysis carried out by the authors (C). Legend: control (light grey) and down-regulated for CtBP1 and CtBP2 (dark grey) relative expression genes.

FIG. 8. Treatment of HeLa cells with Gossypol, Coumermicin and dicumarol reduces proliferation and induce apoptosis. A) HeLa cells were treated for 24 and 48 h with 1 μM of Gossypol, 10 μM Coumermicin A or 100 μM dicumarol. The cells where then analysed by immunofluorescence, with Hoechts to monitor cell number, with antibodies against Ki67 to monitor the proliferation rate, and with antibodies against cleaved PARP1 to monitor the fraction of apoptotic cells. (B-D) Quantitative analysis of cell growth, proliferation and apoptosis. Samples were treated as described in (A) and analyzed by confocal microscopy to evaluate cell number (B), the percentage of proliferating cells (C), and the percentage of apoptotic cells (D). (E) HeLa cells were treated for 48 h with 1 μM Gossypol, or 10 μM Coumermycin or 100 μM dicumarol in the presence of 20 μM caspase inhibitor Q-VD-OPH. Expression level of CtBPs target gene BIK was analysed by qRT-PCR.

### MATERIALS AND METHODS

Unless otherwise specified, all of the reagents were from Sigma-Aldrich. [³²P]-β-NAD⁺ was from Perkin Elmer. The anti-BFA antibody was provided by dr. Vasiliki Lalioti, University of Madrid, Spain. The anti-BARS antibody (BC3) was produced as described previously (9). E-cadherin and vinculin were analized by Western Blotting using Epithelial-Mesenchymal Transition (EMT) antibody sampler kit (Cell Signaling Technology, Cat. N. 9782). Cell-culture reagents were from Gibco/Invitrogen. The TransIT-LT1 reagent was from Minis Bio LLC. Control (CD38 (−)) and CD38 (+) HeLa cells were kindly provided by Prof Antonio De Flora, University of Genoa, Genoa, Italy. GST-E1A (25), His-BARS (26), GST-14-3-3γ (9) and GST-PAK1 (10) were purified as previously described. Rat-brain cytosol and total membranes were prepared as described previously (27). HeLa cells were grown as previously described (12).

### Immunoprecipitation, Cell Fractionations and Transfections.

Total membrane fractions were prepared starting from confluent HeLa cells, which were washed three times with ice-cold phosphate-buffered saline, and mechanically detached in 800 HEPES buffer (20 mM HEPES at pH 7.4, 1 mM EDTA, 250 mM sucrose). The cells were recovered and then sonicated on ice three times for 15 s; unbroken cells were removed by centrifugation at 500×g for 5 min. The resultant supernatants were ultra-centrifuged for 1 h at 100,000×g, with the pellets representing the total membrane fraction. The total membrane fractions were then resuspended in 20 mM HEPES at pH 7.4, containing 1 mM EDTA and protease inhibitors, and stored at −80° C.

HeLa cells were transiently transfected with cDNAs coding for wild-type YFP-BARS or its point mutant His304Ala, using TransIT-LT1 transfection reagent (Minis), according to the manufacturer instructions. For immunoprecipitation experiments, the cells were washed three times in ice-cold phosphate-buffered saline and lysed using 1% Triton lysis buffer (50 mM Tris-HCl at pH 7.4, 150 mM NaCl, 5 mM MgCl₂, 1 mM DTT, 5 mM EGTA, 1% [w/v] Triton X-100, supplemented with protease inhibitor cocktail). Total lysates were centrifuged (15000×g, 10 min, 4° C.) and then incubated with an anti-BARS antibody. After an overnight incubation, 25 μl Protein-A Sepharose beads (Amersham) were added, with an incubation for an additional 1 h at 4° C. The suspensions were then centrifuged for 5 min at 500×g, and the supernatants were recovered. The matrices were washed 5 times and the bound proteins were eluted by boiling the samples for 10 min in 80 μl SDS sample buffer. The immunoprecipitated proteins were separated on 10% SDS-PAGE gels, transferred onto nitrocellulose, and subjected to Western blotting.

### Western Blotting

The nitrocellulose filters were incubated in blocking solution (5% milk in PBS) for 1 h at RT, and then with the primary antibody (diluted 1:500) in the antibody dilution buffer (PBS containing 1% BSA and 0.1% Tween-20, TTBS). After 2-3 h of incubation at RT the antibody was removed and the filters washed in TTBS twice, for 10 min each. The filters were next incubated for 1 h with the appropriate horse radish peroxidase (HRP)-conjugated secondary antibody diluted 1:5000 in antibody dilution buffer, and washed twice in TTBS, for 10 min each, and once in TBS, for 3 min. After washing, the strips were incubated with the ECL reagents, according to the manufacturer instructions, for ECL-based detection by a short exposure to blue-light sensitive autoradiography films.

### BAC Synthesis

Total membranes from rat brain were incubated in the presence of BFA (80 μg/ml), NAD (5 mM) and 0.01 μCi/μl [³²P]-NAD⁺ in metabolite buffer (20 mM Tris-HCl at pH 7.0, 50 mM NaCl), at 37° C. for 2 h. The samples were then centrifuged at 40,000×g at 4° C. for 45 min, and the collected supernatant was filtered using an ultrafiltration apparatus (Amicon) (molecular weight cut-off, 10 kDa). Then, the flow through was extracted twice in two volumes of MeOH/CHCl₃ (1:2, v/v), and the aqueous phase containing the metabolite of interest was lyophilized and stored at −20° C. for further purification.

### BAC Purification

BAC purification was performed using a Waters 2487 Binary Pump HPLC system equipped with a Waters 1525 Dual 1 Absorbance Detector (Kontron HPLC Pump 420), and an ACS UV-Vis detector (model 750/11/AZ) set at 254 nm. The lyophilized BAC fraction was resuspended in 1 ml buffer A (10 mM KH₂PO₄ containing 2.5 mM tetrabutyl ammonium chloride) and loaded onto a semi-preparative C18 reverse-phase column (25×250 mm; pore size, 10 μm) (Viosfer) equilibrated in buffer A. The elution was carried out at a flow rate of 2.5 ml/min using a non-linear gradient of buffer B (40% buffer A, 60% methanol): Time (T), 0 min (100% A, 0% B); T 20 min (50% A, 50% B); T 30 min (0% A, 100% B); T 35 min (0% A, 100% buffer B); T 40 min (100% A, 0% buffer). The collected fractions were then analyzed in ADP-ribosylation assays, as described above, and those positive were pooled, supplemented with sucrose (10 mM), and lyophilized. The sample was then resuspended in 1 ml water and further purified using the same C18 reverse-phase column equilibrated with water. The elution was performed at a flow rate of 2.5 ml/min using a non-linear gradient of buffer B (80% methanol, 20% water): T 0 min (100% A, 0% B); T 10 min (77.5% A, 22.5% B); T 13 min (50% A, 50% B); T 23 min (0% A, 100% B); T 35 min (100% A, 0% B). The fractions containing the purified metabolite were pooled, supplemented with 10 mM sucrose and lyophilized. The sample was resuspended in 20 mM Hepes, pH 7.2 (BAC final concentration, 100 μM), and was then aliquoted and stored at −20° C. for further analysis.

### NAD Competition Assay

Five μg of recombinant BARS were incubated for 2 h at 37° C. in 100 μl buffer (20 mM HEPES at pH 7.4, 25 mM NaCl and 10 μM [³H]-NAD⁺ (specific activity: 1 μCi/μmol)) in the presence of a range of concentrations (from 0.01 to 100 μM) of the molecules listed in table 1. At the end of the incubation BARS was recovered by trapping it to nitrocellulose using a Dot-Blot apparatus (Bio-Rad Laboratories, UK) according to manufacturer instruction. The samples were washed three times in ice-cold HEPES buffer (20 mM, pH 7.4). The amount of radiolabelled NAD bound to BARS was measured using a BetaImager (BioSpace Lab); the total amount of BARS bound to nitrocellulose was evaluated by red ponceau staining (Sigma-Aldrich) according to manufacturer instructions. Quantitative analyses were performed using GraphPad Prism Software.

### Microarray Analysis

HeLa cells were transfected for 48 h using Lipofectamine 2000 (Life Technologies) with 100 nM of non-targeting or with a single siRNA sequence that targets both CtBP1 and CtBP2 (described in Bergman et al., Molec and Cell Biology, 29:16, 4539-4551). The efficacy of the depletion was assessed by western blotting with an anti-CtBP antibody (anti-CtBP1: BD Transduction lab, cod: 612042; anti-CtBP2: Santa Cruz Biotech, cod. Sc-5966) and normalized with an anti-GAPDH antibody (AbD Serotech, cod. 4699) (as a reference). Total mRNA from HeLa cells was extracted using RNeasy kit (Qiagen) according to the manufacturer instructions. The mRNA from three independent experiments was collected and analysed using a GeneChip Human Genome U133A 2.0 Array (Affymetrix) in outsourcing (Coriell Institute for Medical Research; Camden, N.J., USA). The genes that were found upregulated in the more than 1.5 times after CtBP depletion were subjected to a Gene Ontology (GO) Enrichment Analysis performed trough DAVID Bioinformatics Resource (http://david.abcc.ncifcrf.gov/). Genes that most up-regulated and displayed an enrichment in GO categories corresponding to regulation of cell proliferation and regulation of cell death were validated trough RT-PCR analysis. The following genes were selected: Tight junction protein ZO-1 (ZO1) (NCBI Accession numbers: protein: AAA02891.1, nucleotide: HF548122.1); Glioma pathogenesis-related protein 1 (GLIPR1) (NCBI Accession numbers: protein: P48060.3, nucleotide: NM_006851.2) and Keratin, type I cytoskeletal 17 (KRT17) (NCBI Accession numbers: protein: Q04695.2, nucleotide: NM_000422.2).

### Cell Viability Assay

HeLa cells were seeded in glass-bottom 96 wells plates (Greiner Bio-One; 2000 cells/well) and grown in culture medium for 48 h in the presence of various concentrations of the molecules (ranging from 0.01 to 100 μM). At the end of the incubation the cells were fixed with 4% paraformaldehyde (Electron Microscopy Sciences, Hatfield, Pa.) for 10 min at room temperature. The blocking reagent (0.5% bovine serum albumin, 0.1% saponin, and 50 mM NH₄Cl) was then added to the cells for 20 min. The cells were then washed with phosphate-buffered saline and incubated with 2 μg/ml Hoechst 33342mto label the DNA. The cell number was evaluated using an Olympus Microscopy system (Scan̂R) fluorescence microscope according to the manufacturer instructions. Quantitative analyses were performed using a GraphPad Prism Software.

### Effects on the Expression of CtBP Target Genes.

HeLa cells were seeded in 24 wells (Gibco, 30.000 cells/well) and grown in culture medium for 48 h and 96 h in the presence of various concentrations of the molecules (ranging from 0.01 to 100 μM). At the end of the incubations part of the samples was analyzed by western blot. To this purpose, the samples were washed with ice-cold PBS and lysed in 100 μl of SDS sample buffer. The samples were then separated on 8% SDS-PAGE gels, transferred onto nitrocellulose, and subjected to Western blotting using an antibody against Zonula Occludens (anti ZO1; Cell Signaling N. Cat. 5406) and an antibody against GAPDH (AbD Serotec) as a reference. The anti ZO1 antibody was used at a 1:1000 dilution; the anti GAPDH antibody was used at an 1:100000 dilution. The nitrocellulose membranes were subjected to ECL (as described above). The freeware ImageJ software (http://imagej.nih.gov) was used for quantitative analysis.

Part of the samples was also analysed by RT-PCR (as described below) probing for the mRNA levels of ZO1, GLIPR1 and KER17. The samples were normalized with the mRNA levels of Glyceraldehyde-3-phosphate dehydrogenase (GAPDH). A treatment was considered as positive if induced a doubling of the normalized mRNA levels of a CtBP target gene.

### Quantitative Real-Time PCR

Total RNA from HeLa cells was extracted using RNeasy kit (Qiagen) according to the manufacturer instructions. Strands of cDNA were synthesized using a cDNA reverse transcription kit (Qiagen) starting from 1 μg of total RNA. Quantitative Real-time PCR measurements were performed using the Light Cycler 480 Real-Time PCR System (Roche). Each sample was measured in duplicate and the data were analysed with the C_{T} method (2^{-DDCT}) for comparing relative expression results. Resting cells were considered the reference sample, and Homo sapiens hypoxanthine phosphoribosyltransferase 1 (HPRT1) (NCBI Accession numbers: protein: P55884.3, nucleotide: CR407645.1) served as the house-keeping gene. Statistical analyses were performed using a GraphPad Prism Software.

Primer sequences: HPRT1 forward: tgctgacctgctggattaca (SEQ ID NO: 2), HPRT1 reverse: cctgaccaaggaaagcaaag (SEQ ID NO: 3); BAX forward: ggggacgaactggacagtaa (SEQ ID NO: 4), BAX reverse: ctgtaatcccagctccttgg (SEQ ID NO: 5); P21 foward: gacaccactggagggtgact (SEQ ID NO: 6), P21 reveerse ggcgtttggagtggtagaaa (SEQ ID NO: 7); BIK forward: tcctatggctctgcaattgtca (SEQ ID NO: 8); BIK reverse: ggcaggagtgaatggctcttc (SEQ ID NO: 9); TJP1 (ZO1) forward: caacatacagtgacgcttcaca (SEQ ID NO: 10); TJP1 (ZO1) reverse: 5′ cactattgacgtttccccactc (SEQ ID NO: 11); GLIPR1 forward: ctgtggccactacactcagg (SEQ ID NO: 12); GLIPR1 reverse: agagcgtcaaagccagaaac (SEQ ID NO: 13); KRT17 forward: ggtgggtggtgagatcaatgt (SEQ ID NO: 14); KRT17 reverse: cgcggttcagttcctctgtc (SEQ ID NO: 15); GAPDH forward: atcaccatcttccaggagcga (SEQ ID NO: 16); GAPDH Reverse: gccagtgagcttcccgttca (SEQ ID NO: 17); c-Jun forward tccaagtgccgaaaaaggaag (SEQ ID NO: 18); c-Jun reverse: cgagttctgagctttcaaggt (SEQ ID NO: 19); E-cadherin forward: tgaaggtgacagagcctctggat (SEQ ID NO: 20); E-cadherin reverse: tgggtgaattcgggcttgtt (SEQ ID NO: 21).

### Effects on the Nuclear Localization of CtBP.

HeLa cells were seeded on glass coversilps (50,000 cells/well) and grown in culture medium for 24 h. Then, the cells were incubated for 2 h at 37° C. in the presence of various concentrations of the molecules (ranging from 0.01 to 100 μM). The cells were then fixed with 4% paraformaldehyde (Electron Microscopy Sciences, Hatfield, Pa.) for 10 min at room temperature. The blocking reagent (0.5% bovine serum albumin, 0.1% saponin, and 50 mM NH₄Cl) was then added to the cells for 20 min, followed by a 2-h incubation with an anti BARS antibody. The cells were then washed with phosphate-buffered saline and incubated with secondary antibodies (1:400). The samples were then observed by confocal Microscopy (LSM700, Zeiss).

### Results

### Transfer of ADP-Ribose to BARS is Mediated by Formation of a BFA/ADP-Ribose Conjugate.

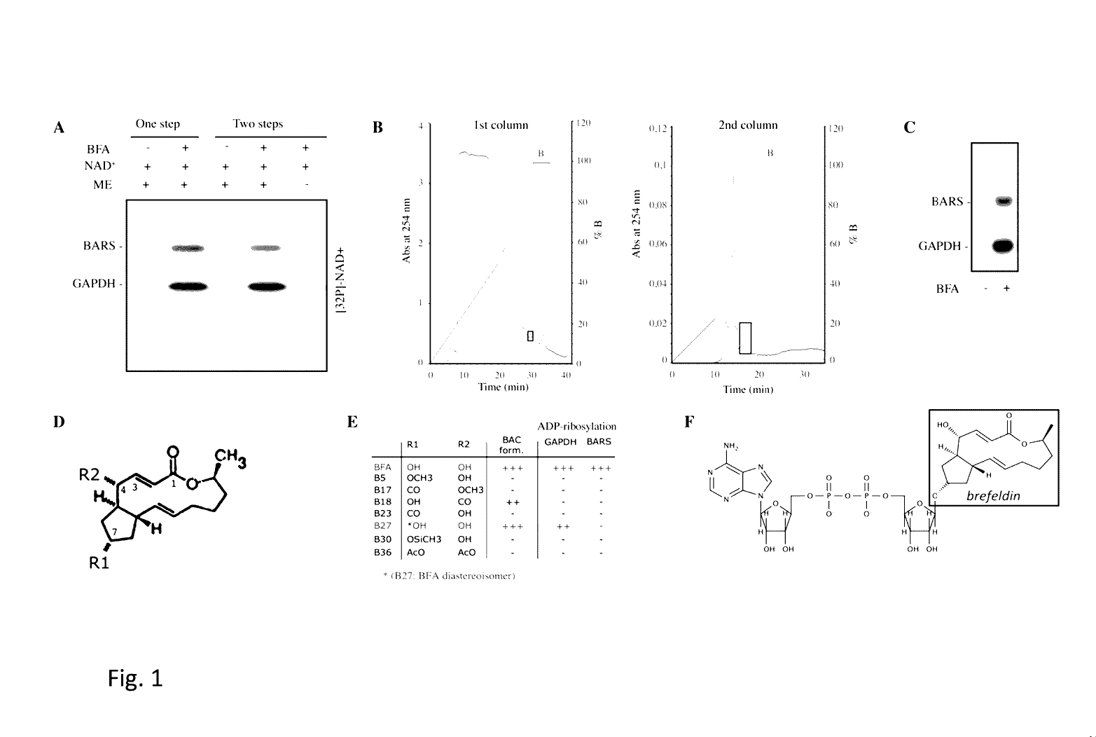

To investigate the molecular mechanisms of the BFA-induced ADP-ribosylation reaction, authors incubated a mixture of rat-brain membranes and cytosol with BFA and [³²P]-NAD⁺ for 1 h at 37° C. (FIG. 1A; one step). As expected, this complete mixture gave rise to ADP-ribosylation of the BARS in the cytosol (5, 6). Next, authors carried out the reaction in two steps, using different mixtures. For the first step, authors incubated the rat-brain membranes (as a source of the enzyme that catalyzes the ADP-ribosylation reaction) with NAD without or with BFA, and NAD and BFA without the membranes. The samples where then centrifuged to remove the membranes, and ultrafiltered (with a 3000-Da cut-off). In the second step, authors mixed this ultrafiltrate with cytosol. Here, the ultrafiltrate that had been generated in the presence of the membranes, NAD and BFA (but not the ultrafiltrate produced in the absence of the membranes or of BFA) resulted in ADP-ribosylation of the cytosolic BARS, as shown by denaturing SDS-PAGE analysis (FIG. 1A; two steps). This indicated that in the presence of BFA and NAD⁺, a membrane-bound enzyme catalyzes the synthesis of a soluble BFA/NAD⁺ derivative that then leads to the modification of BARS.

Authors thus set out to isolate this active derivative by HPLC using ADP-ribosylation of cytosolic BARS for its detection (FIG. 1B, C) (5, 6). The derivative showed an HPLC elution profile that did not match with the elution times of NAD and its known analogs; i.e., ADPR, cyclic ADPR (cADPR), nicotinamide adenine dinucleotide phosphate, nicotinic acid adenine dinucleotide phosphate. Next, to further characterize the active derivative, authors sought to synthesize it using other ADPR-containing molecules as substrates, instead of NAD⁺. Strikingly, the active derivative was synthesized also in the presence of cADPR (albeit with lower efficiency) but not of ADPR. This finding offered a clue towards the determination of the molecular mechanisms of the generation of this BFA/NAD⁺ derivative. Both NAD and cADPR are substrates of a class of enzymes known as ADP-ribosyl cyclases. These cyclases catalyze the conversion of NAD to cADPR through the cleavage of the nicotinamide-ribose glycosidic bond and the formation of an enzyme-stabilized ADP-ribosyl-oxocarbenium ion intermediate (29, 30). This intermediate is a good electrophyle and can react with water, to form ADPR, or intramolecularly, with the N1 atom of the purinic ring of the adenine moiety of NAD⁺, to form cADPR. In addition, the same ADP-ribosyl cyclases catalyze the hydrolysis of cADPR to ADPR via the generation of the same ADP-ribosyl-oxocarbenium ion (3, 31). As BFA has two hydroxyl groups (positions 4 and 7; see FIG. 1D, E), authors hypothesized that these could react with the oxocarbenium ion intermediate. This hypothesis was tested by producing the active derivative through the incubation of [³H]-BFA with [³²P]-NAD⁺, with rat-brain membranes as the source of enzyme, and analyzing the product by HPLC. The results showed that the NAD⁺-derivative contained both [³H] and [³²P] at a 1:1 ratio, indicating that the active derivative is a BFA and ADPR conjugate (BAC). The expected mass of a product composed of BFA and ADPR is 822 Da. To determine the structural features of BFA that are required to induce the formation of BAC, authors tested several BFA analogs that were modified at one or both of the BFA hydroxyl groups (positions 4 and 7; see FIG. 1D, E) (32). Only the BFA analogs carrying a hydroxyl group at position 7, such as B27 (a BFA diastereoisomer) and B18 (FIG. 1D, E), supported the formation of BAC-like derivatives. Importantly, however, these new types of BAC did not react with BARS, which indicated that the structure of BFA is required for BARS ADP-ribosylation (FIG. 1E). The metabolite induced by B27 bound to GAPDH (the other less efficient substrate modified by BAC (26, 27)) to a lesser extent (FIG. 1E). These results indicate that the hydroxyl group at position 7 of BFA reacts with the ADP-ribosyl-oxocarbenium ion intermediate generated by ADP-ribosyl cyclase, to form a BAC (FIG. 1F) that binds to BARS with high efficiency. Other BFA analogs (i.e., those that lack the hydroxyl at position 4 or have an inverted configuration at C7) can induce the formation of BAC-like molecules with distinct features, which do not bind to BARS.

BAC Binds Covalently into the NAD⁺ Binding Pocket of BARS.

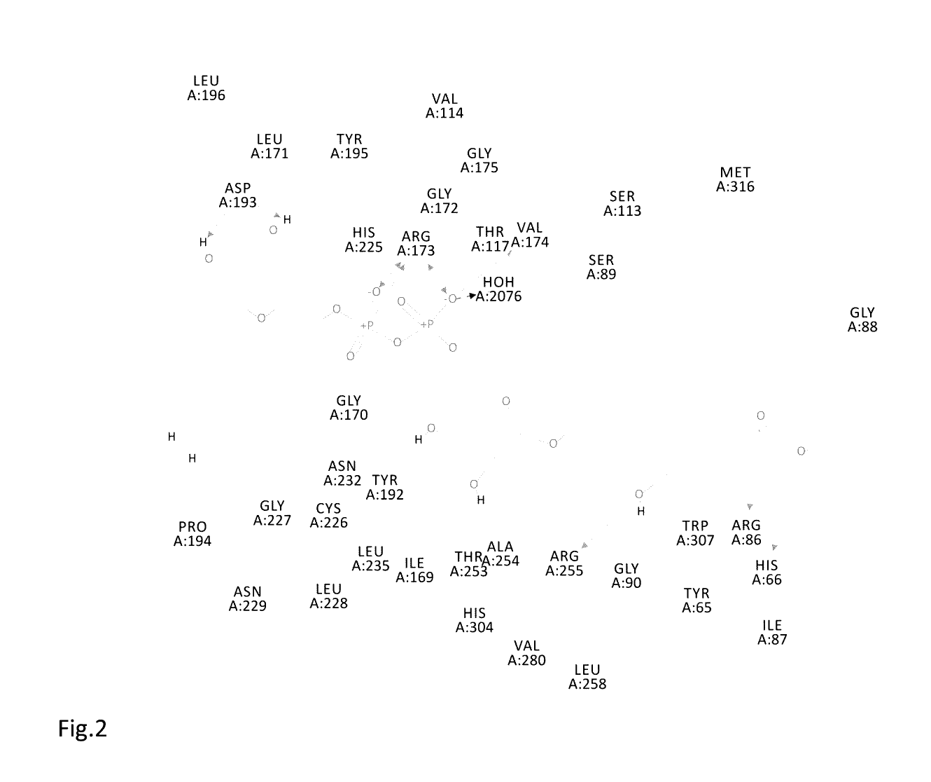

Next, authors focused on the binding of BAC to BARS. This binding must be covalent, as it persists under denaturing SDS-PAGE conditions (FIG. 1A, C). Authors first tested whether the whole BAC molecule binds to BARS, or whether BAC acts as an ADPR donor. To address this point, authors produced BAC using [³H]-labeled BFA. The HPLC-purified [³H]-BAC was incubated with recombinant BARS and examined by SDS-PAGE and a radioactivity Imager. BARS incubated with [³H]-BAC was radiolabeled, indicating that the BARS-bound BAC contains BFA. No signal was detected when BARS was incubated with only [³H]-BFA. Authors also made use of an antibody developed against BFA to further examine whether the whole BAC molecule (i.e., both the BFA and ADPR portions) binds to BARS. If this were the case, the binding of the BFA portion of BAC should be revealed by the immunoreactivity of modified BARS to this anti-BFA antibody. Purified recombinant BARS was incubated with BFA or BAC and treated for immunoblotting. The anti-BFA antibody immunoreacted with BARS after incubation with BAC, confirming the notion that the entire BAC binds to BARS (see below). Notably, this was also the case for the derivative produced using cADPR bound to BARS. To further characterize this reaction, recombinant BARS was incubated with BAC and subjected to both MALDI-TOF mass-spectrometry (MS) and MS/MS and LC-MS analyses. The MALDI analysis showed that, after binding to BAC, BARS has a molecular mass of 822 Da greater than the control BARS protein. This shift corresponds to the calculated mass of BAC, confirming the above predictions that BAC consists of a BFA molecule conjugated with an ADPR, presumably through its hydroxyl group at position 7 (FIG. 1F). Secondly, the fragmentation data indicated that BAC is bound to the His304 residue. In confirmation of this finding, the BARS point mutant His304Ala was not modified by BAC, while several other mutants in the BARS dinucleotide binding site (see below) showed covalent BAC binding levels that were indistinguishable from those of wild-type BARS. Then, to investigate the structural details of the covalent binding of BAC to BARS, authors used a molecular modeling approach and their previous knowledge of the BARS crystal structure, to fit a BAC molecule into the well-characterized NAD(H) Rossman fold in BARS (25), based on the common ADP molecular framework shared by NAD(H) and BAC (FIG. 2). In the crystal structure of the BARS-NAD(H) complex (PDB-code 1HKU), a protein cavity lined by residues Tyr65, His66, Arg86, Gly88, Ser89, Gly90, Asp92, Ser113, Thr117, Arg255, His304, Trp307, Ser313 and Met316 is occupied by the NAD(H) nicotinamide moiety (25). This cavity can host the BFA moiety of BAC (FIG. 2). Analysis of the polarity/hydrophobicity side-chain distribution within the cavity suggested that the orientation of the BFA moiety of BAC would be with its C3 atom in close proximity to the imidazole ring of His304 (FIG. 2). According to this model, the His304 side-chain might be hydrogen bonded to Glu284 (as found in the crystal structure of the BARS-NAD(H) complex), which would assist His304 during a nucleophilic attack on the BFA C3 atom. Furthermore, the BFA carbonylic O1 and hydroxylic O4 atoms would be located in two positively charged pockets that are lined by residues His66/Arg86 and Arg86/Arg255, respectively (FIG. 2). In particular, the Arg86 side-chain is positioned to form a hydrogen bond with the carbonyl group of BFA, thus polarizing the carbon-oxygen bond, while Arg255 binds the carboxylate moiety of BFA, thus helping to position the BFA moiety of BAC correctly in the active site. The BFA plane is further kept in the correct orientation for catalysis by a stacking interaction with the Trp307 side-chain. As the C3 atom of BFA is strongly polarized due to the electron-attractor effect of the nearby conjugated electrophilic carbonyl (lactone) group and of the 4-hydroxy group (34), a rational explanation of the strong and specific binding of BAC to BARS is that the ADPR portion of BAC is involved in recognition and binding to the BARS nucleotide-binding cleft, while the C3 atom of the BFA portion is involved in covalent binding to His304, which can act as an electron donor in a nucleophilic reaction (Michael addition). This hypothesis is supported by the observation that the BAC formed using B18, which is characterized by a diffused conjugated double bond that ranges from C1 to C4, did not bind covalently to BARS (FIG. 1F). Collectively, these findings and the mass spectrometry data, support our computational model of the BARS modification by BAC, and indicate that 1) BAC fits into the BARS Rossman fold with high affinity and specificity, and 2) its BFA portion is involved in covalent binding to His304 through a nucleophilic reaction.

### BAC is Synthesized in Living Cells by the ADP-Ribosyl Cyclase CD38.

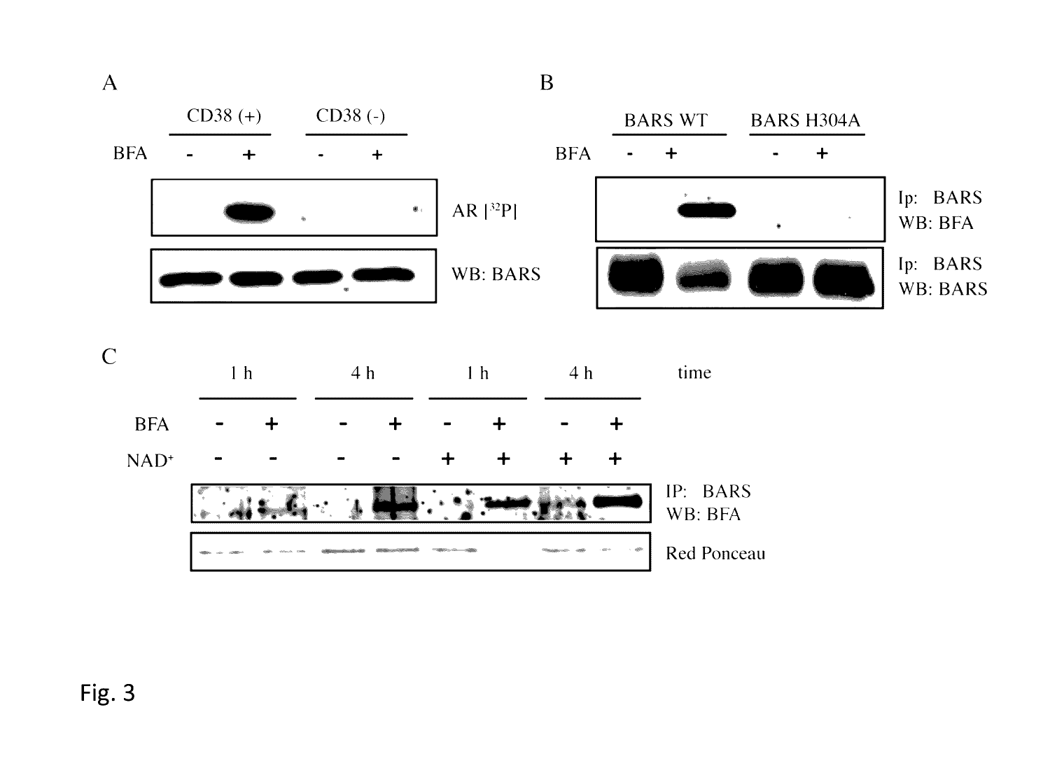

As the above data suggest that BAC is synthesized by an ADP-ribosyl cyclase, authors focused on the membrane-bound ADP-ribosyl cyclase CD38, a mammalian enzyme that is responsible for the synthesis of the Ca²⁺-releasing signaling metabolite cADPR (37). To test for a role of CD38, authors performed the in-vitro assay for BAC formation using whole membrane fractions prepared from control HeLa cells that do not express CD38 (CD38(−)), and from HeLa cells stably-transfected with a vector for CD38 expression (CD38(+)) (38), with analysis by SDS-PAGE and autoradiography. As shown in FIG. 3A, only the membranes obtained from CD38(+) HeLa cells supported the formation of BAC, which demonstrates that this ADP-ribosyl cyclase is indeed involved in the synthesis of BAC. Authors then tested whether BAC formation and binding to BARS by CD38 also occurs in living cells. For this, the CD38(+) HeLa cells were transfected with YFP-BARS or the YFP-BARS His304Ala point mutant, and incubated with NAD⁺ in the absence or presence of BFA. The cells were lysed, YFP-BARS was immunoprecipitated with an anti-BARS antibody, and the samples were subjected to SDS-PAGE and immunoblotting with an anti-BFA antibody. Strikingly, BAC bound to YFP-BARS in CD38(+) Hela cells, while the YFP-BARS His304Ala mutant was not modified, as expected (FIG. 3B). The reaction was very efficient, as more than 90% of the overexpressed wild-type BARS was modified by BAC after 4 h of incubation. Importantly, BARS expressed in the CD38(+) HeLa cells incubated in the absence of exogenously-added NAD (and of other possible sources of NAD⁺, e.g. serum) also showed BAC binding to BARS, although the fraction of the modified BARS and the rate of the reaction were lower than in cells supplemented with NAD⁺ (FIG. 3C). Altogether, these findings show that CD38 can catalyze the formation of BAC in intact cells also in the absence of exogenously added NAD⁺. CD38 is an ectoenzyme and its catalytic domain is localized extracellularly (37-39). It has been proposed that the conversion of extracellular NAD to cADPR and the subsequent cADPR influx is mediated by the juxtaposition of two CD38 monomers, which results in a catalytically active channel (40). Although the extracellular NAD concentration is low in cell culture, this concentration can be locally increased by connexin 43 hemichannels, which translocate NAD to the extracellular space (41, 42). To test whether BAC can be produced outside the cells, control CD38(−) and CD38(+) HeLa cells were incubated with BFA in the presence of NAD⁺. Then, the various media were collected and incubated with recombinant BARS, and protein modification was monitored with the anti-BFA antibody. BARS showed BAC binding when incubated with the medium from the BFA-treated CD38(+) HeLa cells. Thus, BAC can be produced extracellularly. To investigate whether extracellularly generated BAC can cross the plasma membrane, CD38(−) HeLa cells were transfected with YFP-BARS, and treated with BFA and NAD as well as with a recombinant catalytically active soluble portion of CD38, to generate BAC in the medium. As expected, the addition of recombinant CD38 supported the extracellular formation of BAC, but YFP-BARS was not modified, which indicates that the BAC generated in the medium cannot cross the plasma membrane. As a further test, CD38(+) cells were incubated with medium containing purified BAC. Again, no modification of BARS was observed, indicating that extracellular BAC cannot cross the plasma membrane, even in cells that express CD38. Altogether, these data indicate that CD38 is required for the intracellular translocation of BAC, although it might not necessarily transport BAC itself. The exact mechanism of this translocation remains to be determined.

BAC Affects the Oligomerization/Conformation of BARS and Inhibits the Binding of BARS with Interactors Involved in Fission.

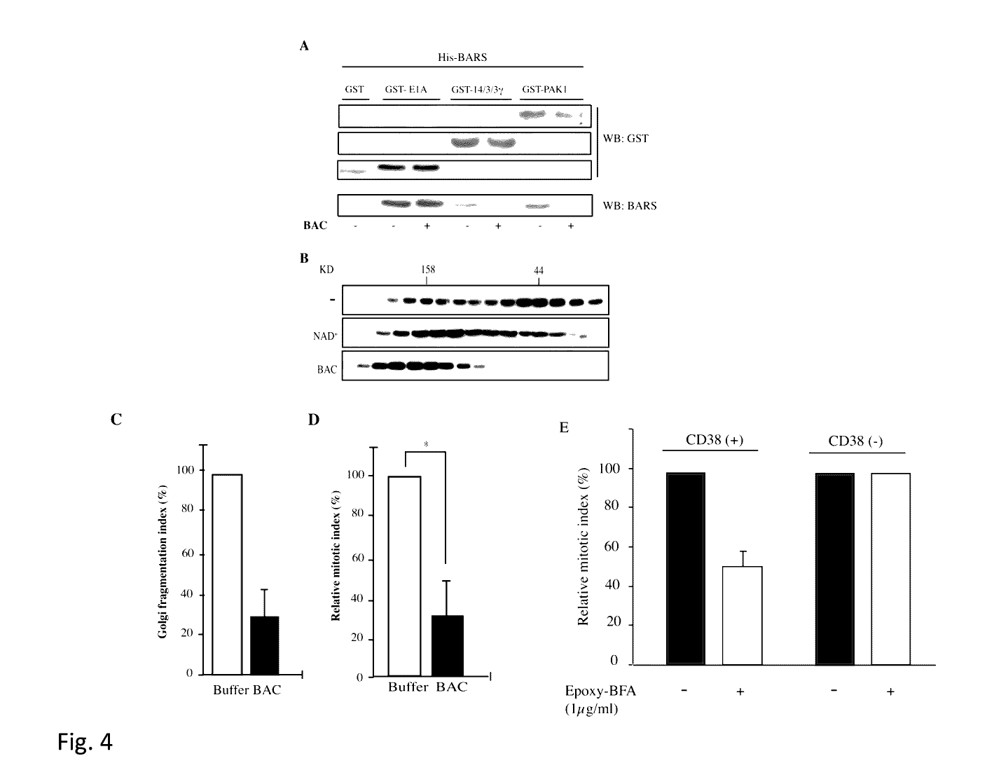

Next, authors investigated the mechanism through which the binding of BAC to BARS affects the activity of BARS (22, 27). It has been proposed that BARS can switch between its nuclear co-repression activity and its membrane-fission activity depending on its binding with two cofactors, NAD(H) and acyl-CoA. Under this model, while NAD(H) promotes a “closed dimeric/tetrameric conformation” and enhances the binding of BARS to cellular and viral transcriptional repressors (25, 43), the binding of palmitoyl-CoA to BARS promotes an “open monomeric conformation” of BARS, which appears to promote membrane fission (11). Recently, 14-3-3γ (NCBI Accession numbers: protein: BAA85184.1, nucleotide: AB024334.1) and the kinase PAK1 (p21 protein (Cdc42/Rac)-activated kinase 1; NCBI Accession numbers protein: AAI09300.1, nucleotide: NM_001128620.1) have been shown to be essential BARS interactors that are involved in the fission of post-Golgi carriers and of macropinosomes (9, 10). Authors investigated whether BAC binding can selectively inhibit BARS interactions with molecular partners involved in membrane fission. To test this hypothesis, authors performed an in-vitro pull-down assay (9). The pre-incubation of immobilized, His-tagged BARS with BAC strongly impaired the ability of BARS to bind to GST-tagged 14-3-3γ and GST-tagged PAK1 (FIG. 4A) (9, 10). Instead, the interaction with E1A, a known cofactor for transcription corepression (25), was not affected by BAC (FIG. 4A). Based on these findings, authors postulated that the covalent binding of BAC to BARS would irreversibly lock BARS in a “closed dimeric/tetrameric conformation”, which would be inactive in fission (11). Authors tested this hypothesis by first examining whether the covalent binding of BAC to BARS can alter the oligomerization state of BARS. Rat-brain cytosol was incubated with control buffer, or with NAD or BAC, and subjected to gel-filtration chromatography. The native protein eluted in two main peaks, which approximately corresponded to the 50 kDa and 170 kDa molecular weight markers, suggesting a BARS conformation that is compatible with an equilibrium between the “open monomeric” and “closed dimeric (and/or tetrameric)” states (27). The incubation with NAD increased the proportion of BARS detected at a molecular mass of 158 kDa (FIG. 4B); after the incubation with BAC, BARS was exclusively found in fractions corresponding to an apparent molecular mass >158 kDa (FIG. 4B). This indicates that the covalent binding of BAC to BARS alters its oligomerization/interaction state by favoring the tetramer, most probably as a result of changes in the BARS conformation (25). As BAC cannot cross the plasma membrane, authors investigated the effects of BAC on the fission-inducing activity of BARS in an in-vitro assay in permeabilized cells that reconstitutes the BARS-dependent mitotic fission and fragmentation of the Golgi complex, a process that is required for entry into mitosis (13). Incubation of permeabilized cells with mitotic cytosol induced fission of the Golgi complex into dispersed fragments, as already reported (FIG. 4C) (13). When BAC was added to mitotic cytosol under conditions that result in exhaustive binding of BARS to BAC, Golgi fission/fragmentation was strongly inhibited (FIG. 4C), which indicated that the covalent binding of BAC to BARS inhibits the ability of BARS to induce mitotic Golgi fragmentation. Then, to test the effect of BAC in living cells, authors induced the ADP-ribosylation of BARS by microinjecting purified BAC in G2-blocked HeLa cells and monitored the effect of this treatment on mitotic entry, which depends tightly on Golgi fragmentation (13). As shown in FIG. 4D, the injection of BAC caused a strong impairment of entry into mitosis, in line with the expected effect of the inhibition of BARS on Golgi fragmentation. In addition, that authors used a BFA analog (10-11 epoxy-BFA) that lacks the effect of BFA on the ARF1 exchange factor and Golgi disassembly but retains the ability to form BAC and ADP-ribosylate BARS. The authors checked the effect of epoxy-BFA on the mitotic index in both CD38 (+) and CD38 (−) live HeLa cells. The results show that epoxy-BFA induces a strong decrease of the number of cells entering mitosis in CD38 (+) but not in CD38 (−) cells (FIG. 4E), in accordance with the inhibitory effect of BAC on mitotic entry. Altogether, these data indicate that the binding of BAC to BARS favors an oligomeric conformation of BARS whereby BARS cannot interact with the proteins necessary for BARS-induced fission, and that this reaction therefore inhibits the ability of BARS to support mitotic fission of the Golgi complex and mitotic entry.

### CtBPs-Mediated Co-Repression Induces Apoptosis and Blocks Cell Proliferation.

CtBPs are NADH-dependent transcriptional repressor that have been linked to tumorigenesis and tumour progression. As NADH binding to CtBPs is required for their corepressor function, authors hypothesized that targeting CtBPs by small molecules that antagonize NADH could provide an antitumor strategy.

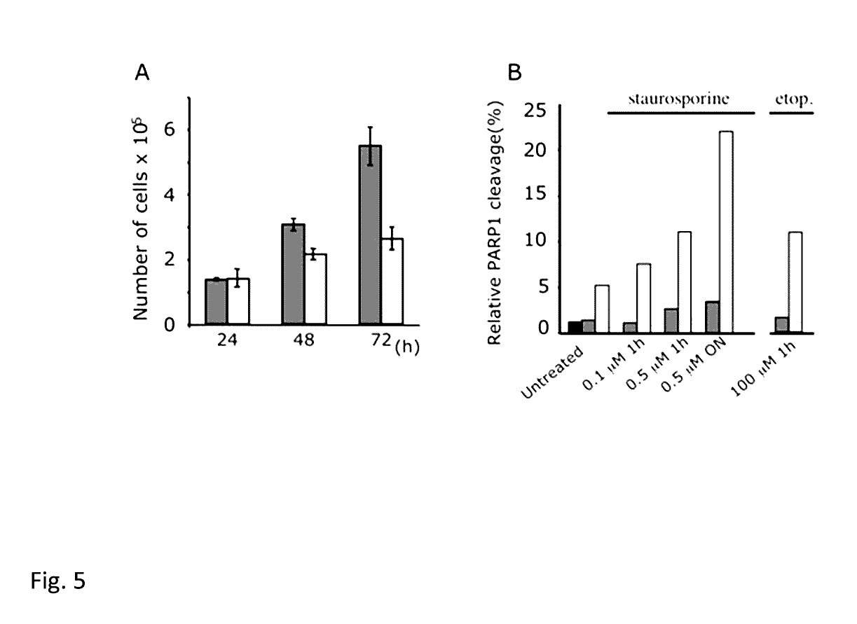

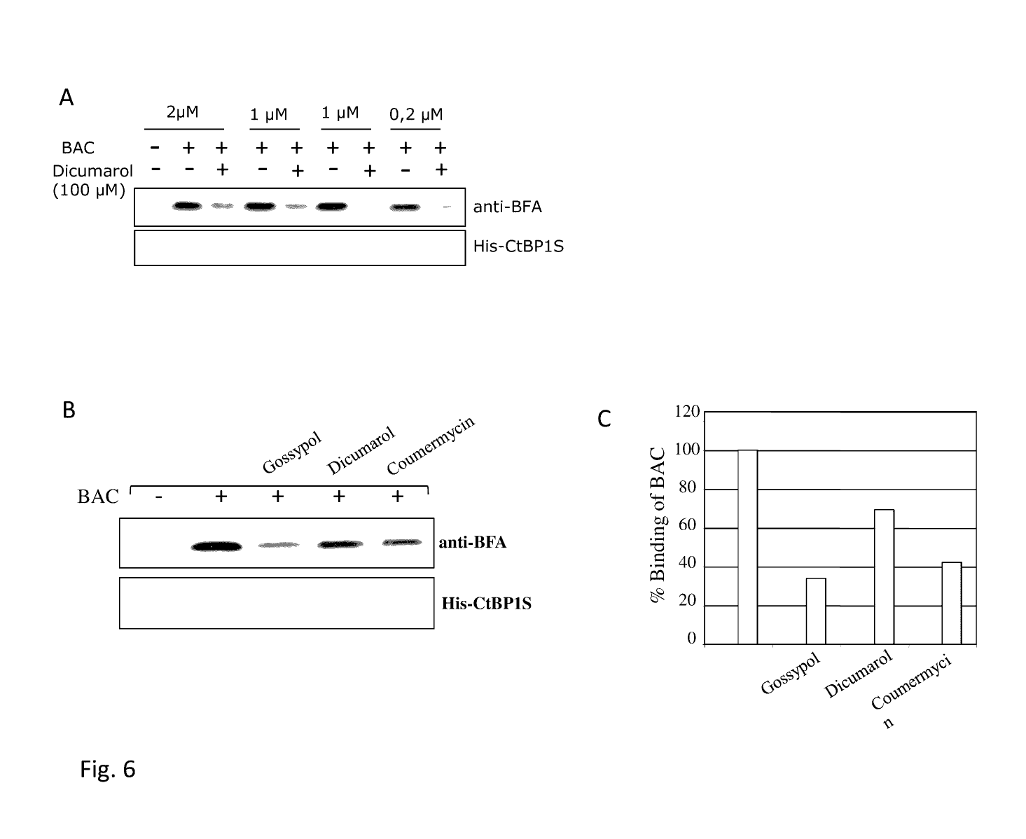

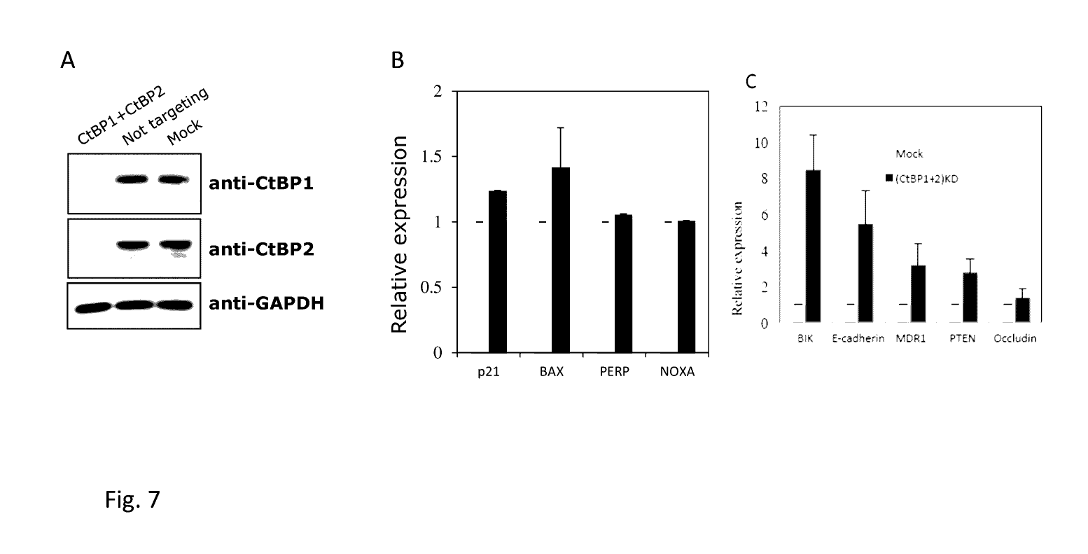

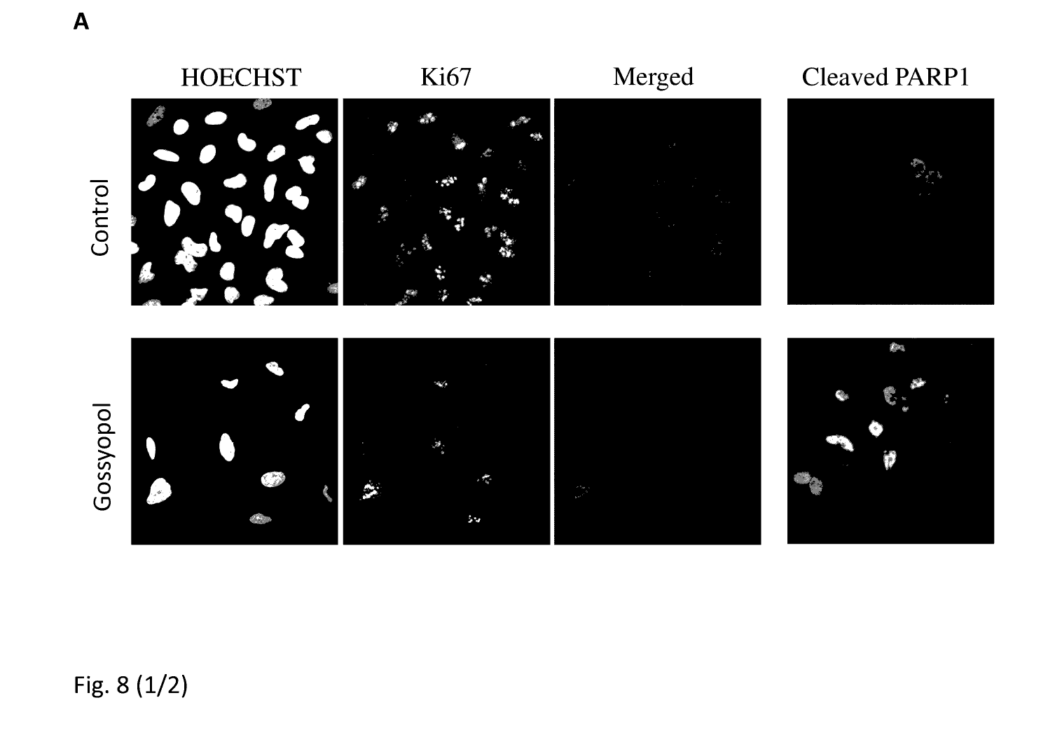

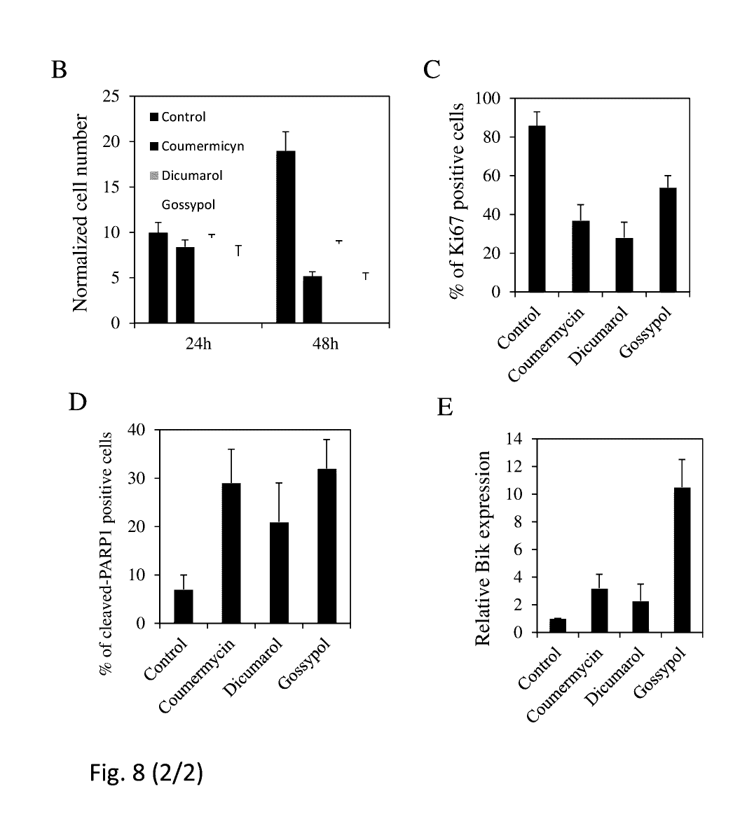

Thus, authors first tested the effect of siRNA-mediated depletion of CtBP1/2 in HeLa cells (human cervical adenocarcinoma). Authors found that CtBP1/2 knock-down caused a strong reduction of cell growth and induced hypersensitisation of cells to apoptotic stimuli (FIG. 5), indicating that reduction of CtBP corepressor activity in HeLa cells produces effects that have a clear relevance to anticancer treatment. To identify lead molecules that bind the Rossman fold and study their cellular effects, authors assayed and demonstrated their capability of inhibiting BAC binding to recombinant CtBP/BARS. By this approach authors identified dicumarol, coumermycin A1, and Gossypol as molecule that can bind to the CtBP Rossmann fold (FIG. 6). Authors then tested whether these compounds could affect cell growth, apoptosis and expression of the pro-apoptotic genes in HeLa cells. Authors first focused on the identification of genes that could act as reporters of CtBP-corepressor activity. Based on literature data and a microarray analysis, authors selected a series of genes to test whether they were under the control of CtBP. By this analysis, Bik was used as the reporter of CtBP co-repressor function (FIG. 7). Then, authors tested the effects of these compounds in HeLa cells by an imaging-based approach for detection of a series of markers of proliferation (Ki67 (NCBI Accession numbers: protein: P46013.2, nucleotide: AJ567757.1)) and apoptosis (cleaved PARP1 (NCBI Accession numbers: protein: P09874.4, nucleotide: BCO37545.1)) and observed a reduction in cell growth, as a consequence of reduced proliferation and increased apoptosis, and a significant increase in expression of the CtBP-target gene Bik (FIG. 8). Therefore, these findings define broad roles for CtBPs in cancer biology and suggest that targeting of CtBPs represents a novel chromatin-based strategy for pharmacological intervention in cancer. In this study, authors describe a novel two-step mechanism that underlies the modification of BARS by BFA, and authors test the functional role of this reaction. The first reaction step is catalyzed by the ADP-ribosyl cyclase CD38 and it leads to the formation of BAC, a BFA-ADP-ribose conjugate. The mechanism of BAC formation is based on the catalytic mechanism of conversion of NAD⁺ to cADPR. This involves cleavage of the NAD⁺ nicotinamide-ribose bond and the subsequent formation of an enzyme-stabilized ADP-ribosyl-oxocarbenium ion intermediate with good electrophilic properties, which reacts with the hydroxyl groups in position 7 of BFA to form BAC. Notably, again in common with the mechanism of conversion of NAD⁺ to cADPR, the synthesis of BAC occurs at the external cell surface, wherefrom BAC is translated, during synthesis, into the cell cytosol, as has been proposed to occur for cADPR synthesis and influx (40). The second step is the covalent binding of BAC into the BARS NAD(H)-binding pocket (the Rossmann fold) (25). As shown in the model in FIG. 2, a compelling explanation of this reaction is that the C3 atom of BFA in BAC is positioned in close proximity of the imidazole ring of His304 of the BARS binding pocket, and it undergoes nucleophilic attack by His304. The reaction is assisted by the hydrogen bond between His304 and Glu284 (FIG. 2). Notably, this binding mechanism is in agreement with the known similarities between BARS and D2-hydroxy acid dehydrogenases (25), where the structurally equivalent His/Glu(Asp)/Arg triad functions as the center for substrate binding and dehydrogenase activity. Here, the His residue is postulated to be the acid/base catalyst, with the Glu/Asp residue helping to lower the His pKa to stabilize it in an unprotonated state. The Arg residue is proposed to polarize the substrate 2-hydroxyl group for catalysis (25). This reaction between BAC and BARS is exquisitely selective, as BAC binds covalently exclusively to the Rossman fold of BARS, but not to that of other dehydrogenases, with the partial exception of GAPDH, where the reaction is orders of magnitudes less efficient. Such a remarkable selectivity suggests that this reaction might have a role in the toxicity of BFA, perhaps in cell types or organisms expressing high levels of CD38 or other similar ADP-ribosyl cyclases. Indeed, the modification of BARS by BAC can impair the fission-inducing activity of BARS that is required for mitotic Golgi fragmentation, an effect that may result in a potent and prolonged block in G2 of the cell-cycle, and eventually in cell apoptosis (12, 13). Perhaps more important, the fact that BFA can lead to the covalent binding of BAC to BARS has implications for cancer treatment. Based on the structural data that are now available on the mechanism of this binding (this study) and on the binding of BFA to the ARF GTPase exchange factor (44), it is now possible to design BFA analogs with increased selectivity towards the formation of BAC-modified BARS, and with no or strongly reduced effects on the ARF GTPase exchange factor. This provides a strategy for the generation of BFA analogs with selective pharmacological effects on the cell cycle. Such analogs would be relevant for the treatment of tumors that are characterized by high levels of CD38 expression, and hence high rates of BAC synthesis, such as multiple myelomas (45, 46).

### Modelling, Synthesis and Screening of Other Pharmacological Modulators of BARS.

The BARS-BAC complex herein revealed was used for molecular modelling and virtual docking studies. The compounds used for the virtual docking on the BARS protein were selected from three main database of 3D ligand structure: (i) Comprehensive Medicinal Chemistry (CMC, 9139 compounds); (ii) MDDR Database (210910 compounds); (iii) and from the Integrity database. Only the NADH-dependent-enzyme competitive inhibitors were retained. A second virtual library was performed using the whole KEGG COMPOUND Database. This approach led to select about 300 compounds for biological tests, that are in progress. Of these, only 28 were commercially available and are listed on tables I-III. Through the BAC competition assay, the present inventors have found that all the molecules were able to compete with BAC for the binding to recombinant BARS. Thus, these experiments confirmed that the compounds selected trough the docking studies were able to bind the Rossman fold. To rank the molecules according to their affinity for BARS, the inventors set up a new assay for determining the capability of the 28 candidate molecules to compete for the binding of NAD to BARS. To this purpose, radiolabeled NAD and recombinant BARS were incubated for 2 h in the presence of a range of concentrations (from 0.01 to 100 μM) of the molecules. At the end of the incubation BARS was recovered by trapping it to nitrocellulose using a dot blot apparatus, and the radiolabelled NAD bound to BARS was evaluated by measuring the radioactivity using a BetaImager (BioSpace Lab). This investigation has led to select 13 molecules (C1, C2, C4, C6, C7, C10, C11, C12, C25, C26, C27, C28, C29) in addition to CA (Gossypol), CB (Coumermycin A1) e CC (Dicoumarol), as the best competitors for the binding of NAD to BARS, thus acting as inhibitor (see Table I). In particular, compound C7 showed an affinity of about 1 microM. All the other remaining 12 molecules were capable of displacing NAD only when used at the highest concentration (100 μM).

### Functional Studies

### A) Test on Cell Viability.

Hela cells have been grown on 96 multiwell plates for 48 h in the presence of variable concentrations of all the compounds selected by virtual docking (concentration range: from 0.01 to 100 μM). At the end of the incubation the cells have been fixed, stained with Hoechst to label the DNA and analysed using a fluorescence microscope for the automated images acquisition and analysis (Olympus ScaR). This investigation has revealed that 18 (C1, C4, C5, C6, C8, C9, C10, C12, C13, C15, C16, C17, C18, C21, C26, C27, C28, C29) molecules did not affect (or had minor effects) cell viability even when used at 100 microM. An EC50 was calculated for the remaining molecules. Among these, the compounds C7 and C11 showed an EC50 of 50 and 10 microM, respectively

### B) Effects on CtBP Co-Repressive Function.

As inventors' preliminary investigation revealed that the set of genes under the control of CtBPs is cell-type specific, a microarray analyses was performed to identify genes that are up- or down-regulated when the expression of CtBPs is silenced in HeLa cells. Based on the result of the microarray, inventors focussed on a cluster of genes that was highly upregulated. The CtBPs-dependent modulation of the expression of the majority of these genes, including GLIPR1, c-Jun (NCBI Accession numbers: protein: P05412.2, nucleotide: NM_002228.3), E-cadherin (NCBI Accession numbers: protein: P12830.3, nucleotide: Z35402.1), Keratin 17, Zonula Occludes-1, vinculin (NCBI Accession numbers: protein: AAB21657.1, nucleotide: M33308.1) and others was validated by qRT-PCR and/or Western Blotting analysis. Thus, this analysis led to the identification of several genes that are up-regulated in knock-down HeLa cells and that are involved in regulation of epithelial-to-mesenchimal transition, cell death and response to stress. Finally, HeLa cells were incubated for 2 and 4 days in the presence of a range of concentrations of all the molecules identified by the docking studies molecules. The highest concentrations used in these experiments were always below the EC50 identified through the viability studies. The effect on CtBP co-repressive function was evaluated by western blot analysis of the total amounts of Zonula Occludens, which is a CtBP-target genes identified by our microarray studies. This analysis revealed 12 out of the 25 compounds (molecules C1-C19, C21, C25-29), i.e. C1, C4, C7, C8, C9, C11, C13, C15, C25, C26, C27 and C28, were able to increase the protein levels of Zonula Occludes. Based on these results and on the capability of the molecules to displace NAD, a subset of 4 molecules (C7, C11, C27, C28) was further investigated for its capability of inhibiting CtBP corepresessive function by analysing through qRT-PCR the effects of a range of concentrations administered for 2 or 4 days. This analysis revealed that compounds C7 and C11 were able to induce an increase of the mRNA levels of three CtBP-target genes (GLIPR1, Ker17 and ZO-1). This effect was detectable after 2 d of incubation using a concentration of 1 microM for compound C11, 25 microM for compound C7 and of 2.5 μM for compound C27, while no effect was detected for compound 28 (see Table I). Therefore, these data support the finding that the Rossmann fold of CtBPs can be exploited for the design of anti-tumoral molecules that affect the transcription-related functions of CtBP/BARS. At least C7 and C11 are expected to inhibit tumour progression in specific types of cancer (breast, colon, lung and melanoma), which have been described to depend on CtBP co-repressive function for their survival (Straza et al, Cell cycle, 2010; Di L J, Byun J S et al, Nature communication, 2013).

C) Translocation of CtBP1 from the Nucleus.

Next, inventors investigated if the selected molecules affect the cellular localization of CtBP1. CtBP1 can switch between its nuclear co-repression activity and its membrane-fission activity depending on several factors, including its cofactors NAD(H). Under this model, while NAD(H) promotes a “closed dimeric/tetrameric conformation” and enhances the binding of BARS to cellular and viral transcriptional repressors, thus forcing a nuclear localization, the displacement of NAD by a competing molecules should cause the disruption of the dimer. This disruption should be revealed by a translocation of CtBP1 form the nucleus to the cytoplasm and with an enhancement of its fissioning activity of CtBP1. Thus, Hela cells have been grown on coverslips and treated for 2 h in the presence of variable concentrations of all the compounds selected by virtual docking (concentration range: from 0.01 to 100 microM). At the end of the incubation the cells have been fixed, stained with DAPI to label the DNA and analysed using a confocal microscope. This analysis revealed that, in addition to Gossypol and Coumermicin, 5 out of the 28 (C2, C7, C11, C25 and C27) were able to induce CTBP1 translocation, thus providing an evidence that the molecules is effectively displacing NAD from CtBP1.

### D) Regulation of the Fissioning Activity of BARS During Mitosis.

As translocation of CtBP1 into the cytoplasm could be associated to an increase of the fissioning activity, inventors evaluated this possibility by testing the effect of the molecules on the structure of the Golgi complex. Thus, Hela cells have been grown on coverslips and treated for 2 h in the presence of variable concentrations of all the compounds selected by virtual docking (concentration range: from 0.01 to 100 microM). At the end of the incubation the samples were fixed and stained with a series of Golgi markers to analysed the structure of the organelle by confocal microscopy. This analysis instead revealed that, in addition of Gossypol and Cuomermicin, two compounds (C7 and C11) were able to induce formation of long tubules emanating from the Golgi complex. This effect is diagnostic of an inhibition of the fissioning activity of CtBP1. Thus, the present results indicate that two molecules, namely compounds C7 and C11, are able to cause two effects that are both relevant to anticancer therapies. One is the inhibition of the corepressive function of the CtBPs that results in selective sensitization of cancer cells to stress-inducing agents. Thus, the selected molecules can potentiate the effects of chemotherapeutic agents. The other is inhibition of CtBP1 fissioning role. As this step is necessary for mitotic Golgi partitioning and cells duplication, the selected molecules are expected to inhibit the G2/M transition of cell cycle and thus reduce cell proliferation.

TABLE I

| | | | | | CtBP target genes | |
|---|---|---|---|---|---|---|
| | | | | | increase (corepressive | |
| | | NAD Displac. | | 50% | activity inhibition) | |
| Code | Common Name | (%) | KI | Viability | Western Blot | RT-PCR |
| CA | Gossypol | 100% | 6.4 μM | 1 μM | | 1 μM |
| CB | Coumermycin A1 | 95% | 14.2 μM | 1 μM | | 10 μM |
| CC | Dicumarol | 20% | | 10 μM | | 100 μM |
| C1 | Warfarin | 15% | | = | 100 μM | |
| C2 | Rotenone | 48% | | 1 μM | = | |
| C3 | STX64 | NO | | 10 μM | = | |
| C4 | Coumachlor | 20% | | = | 10 μM | |
| C5 | Acenocoumarol | NO | | = | = | |
| C6 | Capsaicin | 20% | | = | = | |
| C7 | Epigal. gal. | 100% | 1 μM | 50 μM | 25 μM | 25 μM |
| C8 | | NO | | = | 10 μM | |
| C9 | | NO | | = | 10 μM | |
| C10 | | 30% | | Little | = | |
| | | | | reduction | | |
| C11 | | 70% | | 10 μM | 1 μM | 1 μM |
| C12 | | 23% | | Little | = | |
| | | | | reduction | | |
| C13 | Hydroxycoumarin | NO | | Little | 10 μM | |
| | | | | reduction | | |
| C14 | | NO | | 10 μM | = | |
| C15 | Ononin | NO | | Little | 5 μM | |
| | | | | reduction | | |
| C16 | | NO | | = | = | |
| C17 | Silychristin | NO | | = | = | |
| C18 | Zopolrestat | NO | | Little | = | |
| | | | | reduction | | |
| C19 | | NO | | 1 μM | = | |
| C21 | Thyrot. Rel. Horm. | NO | | = | = | |
| C25 | | 10% | | 1 μM | 0.1 μM | |
| C26 | Risedronate | 30% | | = | 1 μM | |
| C27 | Tiludronic ac. | 20% | | = | 1 μM | 2.5 μM |
| C28 | Aztreonam | 15% | | = | 1 μM | = |
| C29 | Ceforanide | 35% | | = | = | |

In the above table, if the compound has not been tested, no data are indicated.
The symbol “=” means that no effect was show.n
In the two columns named “CtBP target genes increase (corepressive activity inhibition)” the indicated concentrations are the minimum effective concentration and the CtBP target gene tested for gossypol, Coumermycin A1 and Dicumarol through RT-PCR is BIK.

TABLE II
Name, generic (or common) name, CAS Registry number, IUPAC name and SMILE
for each one of the 28 above molecules.

| | | | | CAS | | SMILES (Simplified |
|---|---|---|---|---|---|---|
| | | | Generic | Registry | | Molecular Input Line |
| Number | Code | Name | Name | No. | IUPAC Name | Entry Specification) |
| 1 | CA | | Gossypol | 303-45-7 | 7[8-formyl-1,6,7-trihydroxy-3- | CC1═C(C(═C2C(═C1)C(═ |
| | | | | | methyl-5-(propan-2- | C(C(═C2C═O)O)O)C(C)C)O) |
| | | | | | yl)naphthalen-2-yl]-2,3,8- | C3═C(C═C4C(═C3O)C(═ |
| | | | | | trihydroxy-6-methyl-4-(propan-2- | C(C(═C4C(C)C)O)O)C═O)C |
| | | | | | yl)naphthalene-1-carbaldehyde | |
| 2 | CB | | Coumermycin | 87901-11-9 | 5-Methyl-1H-pyrrole-2-carboxylic | CC1═CC═C(N1)C(═O)O |
| | | | A1 | | acid 3,3-diester with N,N′-bis[7-(6- | [C@@H]2[C@@H](C(OC |
| | | | | | deoxy-5-C-methyl-4-O-methyl- | ([C@H]2OC)(C)C)OC3═C |
| | | | | | alpha-L-lyxo-hexopyranosyloxy)-4- | (C4═C(C═C3)C(═C(C(═O) |
| | | | | | hydroxy-8-methyl-2-oxo-2H-1- | O4)NC(═O)C5═CNC(═C5C) |
| | | | | | benzopyran-3-yl]-3-methyl-1H- | C(═O)NC6═C(C7═C(C(═C |
| | | | | | pyrrole-2,4-dicarboxamide | (C═C7)OC8[C@@H] |
| | | | | | | ([C@@H]([C@H](C(O8)(C) |
| | | | | | | C)OC)OC(═O)C9═CC═C(N9) |
| | | | | | | C)O)C)OC6═O)O)O)C)O |
| 3 | CC | | Dicoumarol | 66-76-2 | 3,3′-Methylenebis(4-hydroxy-2H- | C1═CC═C2C(═C1)C(═C |
| | | | | | 1-benzopyran-2-one) | (C(═O)O2)CC3═C(C4═ |
| | | | | | | CC═CC═C4OC3═O)O)O |
| 4 | C1 | | Warfarin | 81-81-2 | 4-Hydroxy-3-(3-oxo-1- | CC(═O)CC(C1═CC═CC═ |
| | | | | | phenylbutyl)-2H-1-benzopyran-2- | C1)C2═C(C3═CC═CC═ |
| | | | | | one | C3OC2═O)O |
| 5 | C2 | | Rotenone | 83-79-4 | (2R,6aS,12aS)-2-Isopropenyl-8,9- | COc1cc2OC[C@H]3Oc4c |
| | | | | | dimethoxy-1,2,6,6a,12,12a- | 5C[C@@H](Oc5ccc4C |
| | | | | | hexahydro-1-benzopyrano[3,4- | (═O)[C@H]3c2cc1OC)C |
| | | | | | b]furo[2,3-h]-1-benzopyran-6-one | (C)═C |
| 6 | C3 | STX64 | Irosustat | 288628-05-7 | Sulfamic acid 6-oxo-6,7,8,9,10,11- | C1CCC2═C(CC1)C(═O)OC |
| | | | | | hexahydrocyclohepta[c]-1- | 3═C2C═CC(═C3)OS(═O) |
| | | | | | benzopyran-3-yl ester | (═O)N |
| 7 | C4 | | Coumachlor | 81-82-3 | 3-[1-(4-chlorophenyl)-3-oxobutyl]- | CC(═O)CC(C1═CC═C(C═ |
| | | | | | 4-hydroxychromen-2-one | C1)Cl)C2═C(C3═CC═CC═ |
| | | | | | | C3OC2═O)O |
| 8 | C5 | Acenocoumarol | Nicoumalone | 152-72-7 | 4-Hydroxy-3-[1-(4-nitrophenyl)-3- | CC(═O)CC(C1═CC═C |
| | | | | | oxobutyl]-2H-1-benzopyran-2-one | (C═C1)[N+](═O)[O-]) |
| | | | | | | C2═C(C3═CC═CC═ |
| | | | | | | C3OC2═O)O |
| 9 | C6 | | Capsaicin | 404-86-4 | N-(4-Hydroxy-3-methoxybenzyl)-8- | CC(C)/C═C/CCCCC(═O)NC |
| | | | | | methyl-6(E)-nonenamide 8- | C1═CC(═C(C═C1)O)OC |
| | | | | | Methyl-N-vanillyl-trans-6- | |
| | | | | | nonenamide | |
| 10 | C7 | | (-)- | 989-51-5 | (-)-3,4,5-Trihydroxybenzoic acid | C1[C@H]([C@H](OC2═CC |
| | | | Epigallocatechin | | 5,7-dihydroxy-2(R)-(3,4,5- | (═CC(═C21)O)O)C3═CC |
| | | | gallate | | trihydroxyphenyl)-3,4-dihydro-2H- | (═C(C(═C3)O)O)O)OC(═O) |
| | | | | | 1-benzopyran-3(R)-yl ester | C4═CC(═C(C(═C4)O)O)O |
| 11 | C8 | | | | (9R,10S,11S,15R)-13-(1,3- | C1═CC═C2C3C4C(C(C2═ |
| | | | | | dihydroxy-2- | C1)C5═CC═CC═C35)C(═O) |
| | | | | | (hydroxymethyl)propan-2-yl)- | N(C4═O)C(CO)(CO)CO |
| | | | | | 10,11-dihydro-9H-9,10- | |
| | | | | | [3,4]epipyrroloanthracene- | |
| | | | | | 12,14(13H,15H) dione | |
| 12 | C9 | | | | 6-methyl-2-[(E)-(thiophen-2- | CN1CCC2═C(C1)SC(═C2C |
| | | | | | ylmethylidene)amino]- | (═O)N)N═CC3═CC═CS3 |
| | | | | | 4H,5H,6H,7H-thieno[2,3- | |
| | | | | | c]pyridine-3-carboxamide | |
| 13 | C10 | | | | (4Z)-2-(4-butoxyphenyl)-4-[(2,4,5- | CCCCOC1═CC═C(C═C1) |
| | | | | | trimethoxyphenyl)methylidene]- | C2═NC(═CC3═CC(═C(C═ |
| | | | | | 4,5-dihydro-1,3-oxazol-5-one | C3OC)OC)OC)C(═O)O2 |
| 14 | C11 | | | | N-(3,4-dichlorophenyl)-4{[(4- | [O—][N+](═O)C1═CC═ |
| | | | | | nitrophenyl)carbamoyl]amino} | C(NC(═O)NC2═CC═C(C═ |
| | | | | | benzenesulfonamide | C2)S(═O)(═O)NC2═CC |
| | | | | | | (Cl)═C(Cl)C═C2)C═C1 |
| 15 | C12 | | RJF-01928 | 353477-88-0 | 2-(4-chlorophenyl)-2-oxoethyl 2- | CC1═C(C(═O)OC2═C1C═C |
| | | | | | {[3-(2,6-dichlorobenzyl)-4-methyl- | C(═C2)OCC(═O)OCC(═O) |
| | | | | | 2-oxo-2H-chromen-7- | C3═CC═C(C═C3)Cl)CC4═C |
| | | | | | yl]oxy}acetate | (C═CC═C4Cl)Cl |
| 16 | C13 | | | | 3,3′-methanediylbis(4-hydroxy-2H- | c1ccc2c(c1)C(═O)C(C(═O) |
| | | | | | chromen-2-one) | O2)CC3C(═O) |
| | | | | | | c4ccccc4OC3═O |
| 17 | C14 | | | | 4-hydroxy-3-(3-oxo-1,3- | C1═CC═C2C(═C1)C(OC2═ |
| | | | | | dihydroisobenzofuran-1-yl)-2H- | O)C3═C(C4═CC═CC═ |
| | | | | | chromen-2-one | C4OC3═O)O |
| 18 | C15 | Ononin | formononetin | 486-62-4 | 3-(4-methoxyphenyl)-7- | COC1═CC═C(C═C1)C1═CO |
| | | | glucoside | | {[(2S,3R,4S,5S,6R)-3,4,5- | C2═CC(O[C@@H]3O[C@ |
| | | | | | trihydroxy-6- | H](CO)[C@@H](O)[C@H] |
| | | | | | (hydroxymethyl)oxan-2-yl]oxy}- | (O)[C@H]3O)═CC═C2C1═O |
| | | | | | 4H-chromen-4-one | |
| 19 | C16 | | | 60-81-1 | 1-(2,4-dihydroxy-6- | OC[C@H]1O[C@@H](OC2═ |
| | | | | | {[(2S,3R,4S,5S,6R)-3,4,5- | C(C(═O)CCC3═CC═C(O) |
| | | | | | trihydroxy-6- | C═C3)C(O)═CC(O)═C2) |
| | | | | | (hydroxymethyl)oxan-2- | [C@H](O)[C@@H](O) |
| | | | | | yl]oxy}phenyl)-3-(4- | [C@@H]1O |
| | | | | | hydroxyphenyl)propan-1-one | |
| 20 | C17 | | Silychristin | 33889-69-9 | 3beta,5,7-Trihydroxy-2alpha-[7- | COC1═C(O)C═CC(═C1)C1 |
| | | | | | hydroxy-2beta-(4-hydroxy-3- | OC2═C(C═C(C═C2O) |
| | | | | | methoxyphenyl)-3alpha- | C2OC3═C(C(O)═CC(O)═ |
| | | | | | (hydroxymethyl)-2,3- | C3)C(═O)C2O)C1CO |
| | | | | | dihydrobenzofuran-5-yl]-2,3- | |
| | | | | | dihydro-4H-benzo[b]pyran-4-one | |
| 21 | C18 | | Zopolrestat | 110703-94-1 | 2-[4-Oxo-3-[5- | C1═CC═C2C(═C1)C(═NN |
| | | | | | (trifluoromethyl)benzothiazol-2- | (C2═O)CC3═NC4═C(S3)C═ |
| | | | | | ylmethyl]-3,4-dihydrophthalazin- | CC(═C4)C(F)(F)F)CC(═O)O |
| | | | | | 1-yl]acetic acid | |
| 22 | C19 | | Estrovis | 152-43-2 | 17alpha-Ethynylestradiol 3- | C[C@]12CC[C@H]3[C@H] |
| | | | | | cyclopentyl ether | ([C@@H]1CC[C@]2(C#C) |
| | | | | | | O)CCC4═C3C═CC(═C4)O |
| | | | | | | C5CCCC5 |
| 23 | C21 | Thyrotropin | protirelin | 117217-40-0 | (2S)-N-[(2S)-1-[(2S)-2- | C1C[C@H](N(C1)C(═O) |
| | | releasing | | | carbamoylpyrrolidin-1-yl]-3-(1H- | [C@H](CC2═CN═CN2)NC |
| | | hormone | | | imidazol-5-yl)-1-oxopropan-2-yl]- | (═O)[C@@H]3CCC(═O) |
| | | | | | 5-oxopyrrolidine-2-carboxamide | N3)C(═O)N |
| 24 | C25 | 17alpha- | Ethinylestradiol | 57-63-6 | 17alpha-Ethynylestradiol | C[C@]12CC[C@H]3[C@H] |
| | | Ethynylestradiol | | | | ([C@@H]1CC[C@]2(C#C) |
| | | | | | | O)CCC4═C3C═CC(═C4)O |
| 25 | C26 | | Risedronate | 105462-24-6 | [1-hydroxy-1-phosphono-2- | OC(CC1═CC═CN═C1)(P(O) |
| | | | | | (pyridin-3-yl)ethyl]phosphonic | (O)═O)P(O)(O)═O |
| | | | | | acid | |
| 26 | | | Tiludronic | 89987-06-4 | [(4-chlorophenyl)sulfanyl- | C1═CC(═CC═C1SC(P(═O) |
| | | | acid | | phosphonomethyl]phosphonic | (O)O)P(═O)(O)O)Cl |
| | | | | | acid | |
| 27 | C28 | | Aztreonam | 78110-38-0 | 2-[(Z)-[1-(2-amino-1,3-thiazol-4- | C[C@H]1[C@H](NC(═O) |
| | | | | | yl)-2-[[(2S,3S)-2-methyl-4-oxo-1- | C(═NOC(C)(C)C(O)═O)c2csc |
| | | | | | sulfoazetidin-3-yl]amino]-2- | (N)n2)C(═O)N1S(O)(═O)═O |
| | | | | | oxoethylidene]amino]oxy-2- | |
| | | | | | methylpropanoic acid | |
| 28 | C29 | | Ceforanide | 60925-61-3 | (6R,7R)-7-[2-[2- | NCC1═CC═CC═C1CC(═O) |
| | | | | | (Aminomethyl)phenyl]acetamido]- | N[C@H]1[C@H]2SCC |
| | | | | | 3-[1-(carboxymethyl)-1H-tetrazol- | (CSC3═NN═NN3CC(O)═ |
| | | | | | 5-ylsulfanylmethyl]-8-oxo-5-thia- | O)═C(N2C1═O)C(O)═O |
| | | | | | 1-azabicyclo[4.2.0]-2-octene-2- | |
| | | | | | carboxylic acid | |

TABLE III
Chemical structures of the above 28 molecules.

| Code | Structure |
|---|---|
| CA | 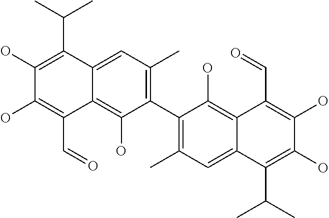 [SMILES: Cc1cc2c(C(C)C)c(O)c(O)c(C=O)c2c(O)c1-c1c(C)cc2c(C(C)C)c(O)c(O)c(C=O)c2c1O] |
| CB | 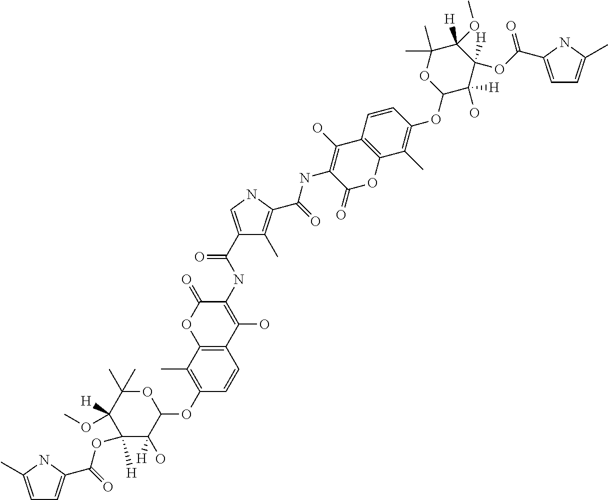 |
| CC | 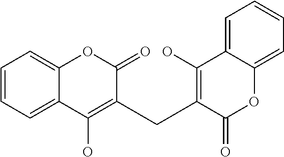 [SMILES: O=c1oc2ccccc2c(O)c1Cc1c(O)c2ccccc2oc1=O] |
| C1 | 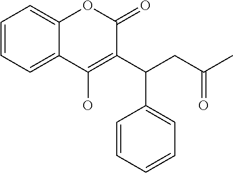 [SMILES: CC(=O)CC(c1ccccc1)c1c(O)c2ccccc2oc1=O] |
| C2 | 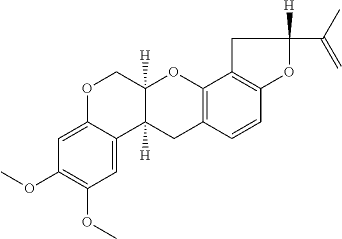 [SMILES: C=C(C)[C@H]1Cc2c(ccc3c2O[C@@H]2COc4cc(OC)c(OC)cc4[C@@H]2C3)O1] |
| C3 | 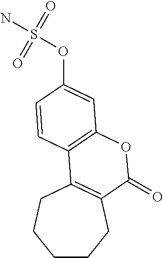 [SMILES: NS(=O)(=O)Oc1ccc2c3c(c(=O)oc2c1)CCCCC3] |
| C4 | 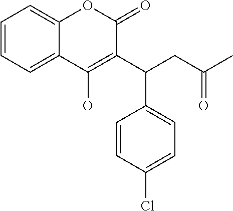 [SMILES: CC(=O)CC(c1ccc(Cl)cc1)c1c(O)c2ccccc2oc1=O] |
| C5 | 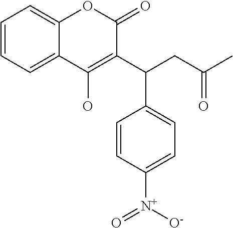 [SMILES: CC(=O)CC(c1ccc([N+](=O)[O-])cc1)c1c(O)c2ccccc2oc1=O] |
| C6 | 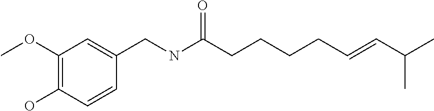 [SMILES: COc1cc(CNC(=O)CCCC/C=C/C(C)C)ccc1O] |
| C7 | 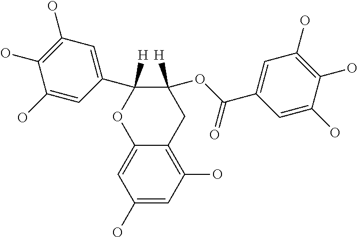 [SMILES: O=C(O[C@@H]1Cc2c(O)cc(O)cc2O[C@@H]1c1cc(O)c(O)c(O)c1)c1cc(O)c(O)c(O)c1] |
| C8 | 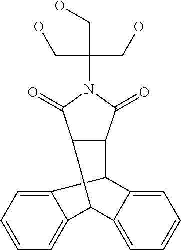 [SMILES: O=C1C2C3c4ccccc4C(c4ccccc43)C2C(=O)N1C(CO)(CO)CO] |
| C9 | 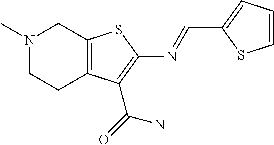 [SMILES: CN1CCc2c(sc(/N=C/c3cccs3)c2C(N)=O)C1] |
| C10 | 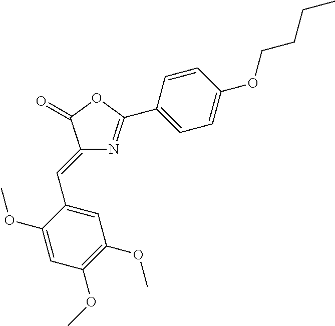 [SMILES: CCCCOc1ccc(C2=N/C(=C\c3cc(OC)c(OC)cc3OC)C(=O)O2)cc1] |
| C11 | 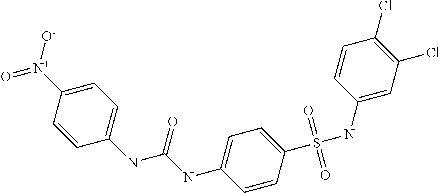 [SMILES: O=C(Nc1ccc([N+](=O)[O-])cc1)Nc1ccc(S(=O)(=O)Nc2ccc(Cl)c(Cl)c2)cc1] |
| C12 | 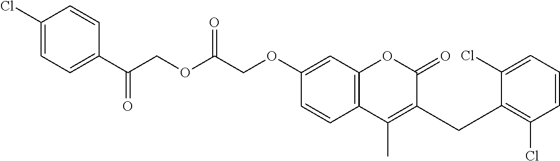 [SMILES: Cc1c(Cc2c(Cl)cccc2Cl)c(=O)oc2cc(OCC(=O)OCC(=O)c3ccc(Cl)cc3)ccc12] |
| C13 | 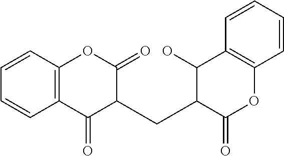 [SMILES: O=C1Oc2ccccc2C(=O)C1CC1C(=O)Oc2ccccc2C1O] |
| C14 | 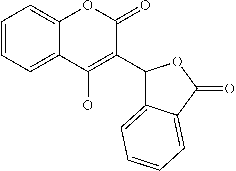 [SMILES: O=C1OC(c2c(O)c3ccccc3oc2=O)c2ccccc21] |
| C15 | 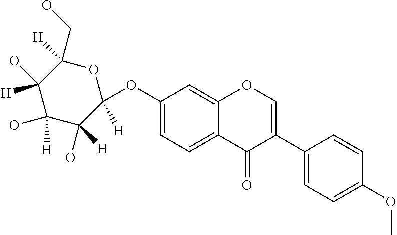 [SMILES: COc1ccc(-c2coc3cc(O[C@@H]4O[C@H](CO)[C@@H](O)[C@H](O)[C@H]4O)ccc3c2=O)cc1] |
| C16 | 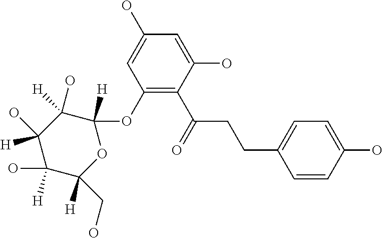 [SMILES: O=C(CCc1ccc(O)cc1)c1c(O)cc(O)cc1O[C@@H]1O[C@H](CO)[C@@H](O)[C@H](O)[C@H]1O] |
| C17 | 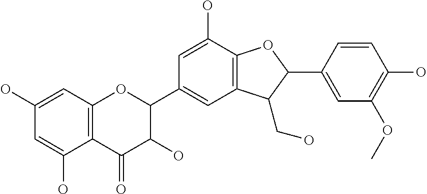 [SMILES: COc1cc(C2Oc3c(O)cc(C4Oc5cc(O)cc(O)c5C(=O)C4O)cc3C2CO)ccc1O] |
| 18 | 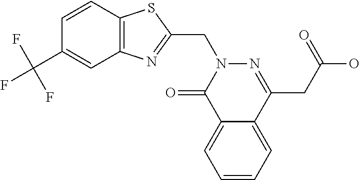 [SMILES: O=C(O)Cc1nn(Cc2nc3cc(C(F)(F)F)ccc3s2)c(=O)c2ccccc12] |
| C19 | 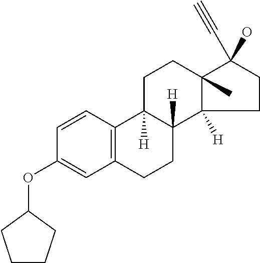 |
| C21 | 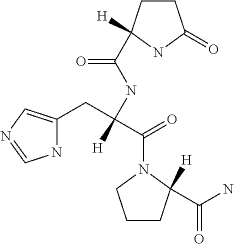 [SMILES: NC(=O)[C@@H]1CCCN1C(=O)[C@H](Cc1cnc[nH]1)NC(=O)[C@@H]1CCC(=O)N1] |
| C25 | 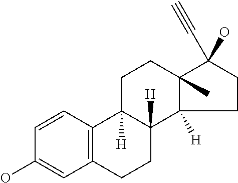 |
| C26 | 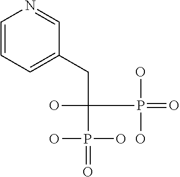 [SMILES: O=P(O)(O)C(O)(Cc1cccnc1)P(=O)(O)O] |
| C27 | 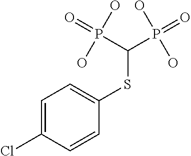 [SMILES: O=P(O)(O)C(Sc1ccc(Cl)cc1)P(=O)(O)O] |
| C28 | 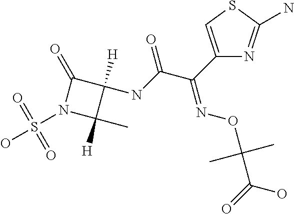 [SMILES: C[C@H]1[C@H](NC(=O)/C(=N/OC(C)(C)C(=O)O)c2csc(N)n2)C(=O)N1S(=O)(=O)O] |
| C29 | 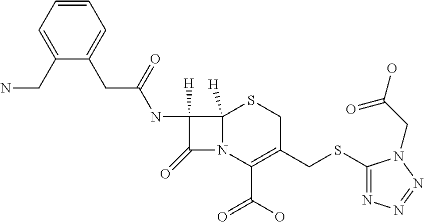 [SMILES: NCc1ccccc1CC(=O)N[C@@H]1C(=O)N2C(C(=O)O)=C(CSc3nnnn3CC(=O)O)CS[C@H]12] |

### REFERENCES

1. Corda D & Di Girolamo M (2003) Functional aspects of protein mono-ADP-ribosylation. Embo J 22(9):1953-1958.
2. Hassa P O, Haenni S S, Elser M, & Hottiger M O (2006) Nuclear ADP-ribosylation reactions in mammalian cells: where are we today and where are we going? Microbiology and molecular biology reviews: MIIBR 70(3):789-829.
3. Koch-Nolte F, Kernstock S, Mueller-Dieckmann C, Weiss M S, & Haag F (2008) Mammalian ADP-ribosyltransferases and ADP-rib osylhydrolases. Front Biosci 13:6716-6729.
4. Di Girolamo M, Dani N, Stilla A, & Corda D (2005) Physiological relevance of the endogenous mono(ADP-ribosyl)ation of cellular proteins. FEBS J 272(18):4565-4575.
5. De Matteis M A, et al. (1994) Stimulation of endogenous ADP-ribosylation by brefeldin A. Proc Natl Acad Sci USA 91(3):1114-1118.
6. Di Girolamo M, et al. (1995) Evidence that the 50-kDa substrate of brefeldin A-dependent ADP-ribosylation binds GTP and is modulated by the G-protein beta gamma subunit complex. Proc Natl Acad Sci USA 92(15):7065-7069.
7. Corda D, Colanzi A, & Luini A (2006) The multiple activities of CtBP/BARS proteins: the Golgi view. Trends Cell Biol 16(3):167-173.
8. Bonazzi M, et al. (2005) CtBP3/BARS drives membrane fission in dynamin-independent transport pathways. Nature cell biology 7(6):570-580.
9. Valente C, et al. (2012) A 14-3-3γ dimer-based scaffold bridges CtBP1-S/BARS to PI(4)KIIIβ to regulate post-Golgi carrier formation. Nature cell biology 14(4):343-354.
10. Liberali P, et al. (2008) The closure of Pak1-dependent macropinosomes requires the phosphorylation of CtBP1/BARS. Embo J 27(7):970-981.
11. Yang J S, et al. (2005) A role for BARS at the fission step of COPI vesicle formation from Golgi membrane. Embo J 24(23):4133-4143.
12. Colanzi A, et al. (2007) The Golgi mitotic checkpoint is controlled by BARS-dependent fission of the Golgi ribbon into separate stacks in G2. Embo J 26(10):2465-2476.
13. Hidalgo Carcedo C, et al. (2004) Mitotic Golgi partitioning is driven by the membrane-fissioning protein CtBP3/BARS. Science 305(5680):93-96.
14. Chinnadurai G (2007) Transcriptional regulation by C-terminal binding proteins. The international journal of biochemistry & cell biology 39(9):1593-1607.
15. Chinnadurai G (2009) The transcriptional corepressor CtBP: a foe of multiple tumor suppressors. Cancer research 69(3):731-734.
16. Bhattacharyya M, Upadhyay R, & Vishveshwara S (2012) Interaction signatures stabilizing the NAD(P)-binding Rossmann fold: a structure network approach. PLoS ONE 7(12):e51676.
17. Lesk A M (1995) NAD-binding domains of dehydrogenases. Current opinion in structural biology 5(6):775-783.
18. Driouich A, Jauneau A, & Staehelin L A (1997) 7-Dehydrobrefeldin A, a naturally occurring brefeldin A derivative, inhibits secretion and causes a cis-to-trans breakdown of Golgi stacks in plant cells. Plant physiology 113(2):487-492.
19. Fujiwara T, Oda K, Yokota S, Takatsuki A, & Ikehara Y (1988) Brefeldin A causes disassembly of the Golgi complex and accumulation of secretory proteins in the endoplasmic reticulum. J Biol Chem 263(34):18545-18552.
20. Donaldson J G, Finazzi D, & Klausner R D (1992) Brefeldin A inhibits Golgi membrane-catalysed exchange of guanine nucleotide onto ARF protein. Nature 360(6402):350-352.
21. Mironov A, et al. (1997) Role of NAD+ and ADP-ribosylation in the maintenance of the Golgi structure. The Journal of cell biology 139(5):1109-1118.
22. Weigert R, et al. (1999) CtBP/BARS induces fission of Golgi membranes by acylating lysophosphatidic acid. Nature 402(6760):429-433.
23. Vassilev L T, et al. (2006) Selective small-molecule inhibitor reveals critical mitotic functions of human CDK1. Proc Natl Acad Sci USA 103(28):10660-10665.
24. Chaitanya G V & Babu P P (2009) Differential PARP cleavage: an indication of heterogeneous forms of cell death and involvement of multiple proteases in the infarct of focal cerebral ischemia in rat. Cell Mol Neurobiol 29(4):563-573.
25. Nardini M, et al. (2003) CtBP/BARS: a dual-function protein involved in transcription co-repression and Golgi membrane fission. Embo J 22(12):3122-3130.
26. Valente C, Spano S, Luini A, & Corda D (2005) Purification and functional properties of the membrane fissioning protein CtBP3/BARS. Methods Enzymol 404:296-316.
27. Spano S, et al. (1999) Molecular cloning and functional characterization of brefeldin A-ADP-ribosylated substrate. A novel protein involved in the maintenance of the Golgi structure. J Biol Chem 274(25):17705-17710.
28. Paulik M, et al. (1999) Drug-antibody conjugates with anti-HIV activity. Biochem Pharmacol 58(11):1781-1790.
29. Sauve A A, Munshi C, Lee H C, & Schramm V L (1998) The reaction mechanism for CD38. A single intermediate is responsible for cyclization, hydrolysis, and base-exchange chemistries. Biochemistry 37(38):13239-13249.
30. Egea P F, et al. (2012) Insights into the mechanism of bovine CD38/NAD+glycohydrolase from the X-ray structures of its Michaelis complex and covalently-trapped intermediates. PLoS ONE 7(4):e34918.
31. Kotaka M, et al. (2012) Structural studies of intermediates along the cyclization pathway of Aplysia ADP-ribosyl cyclase. J Mol Biol 415(3):514-526.
32. Klausner R D, Donaldson J G, & Lippincott-Schwartz J (1992) Brefeldin A: insights into the control of membrane traffic and organelle structure. The Journal of cell biology 116(5):1071-1080.
33. Dani N, et al. (2009) Combining affinity purification by ADP-ribose-binding macro domains with mass spectrometry to define the mammalian ADP-ribosyl proteome. Proc Natl Acad Sci USA 106(11):4243-4248.
34. Bruning A, et al. (1992) Brefeldin A binds to glutathione S-transferase and is secreted as glutathione and cysteine conjugates by Chinese hamster ovary cells. J Biol Chem 267(11):7726-7732.
35. Weigert R, et al. (1997) Characterization of chemical inhibitors of brefeldin A-activated mono-ADP-ribosylation. J Biol Chem 272(22):14200-14207.
36. Ma Q, Wang R, Yang C S, & Lu A Y (1990) Expression of mammalian DT-diaphorase in Escherichia coli: purification and characterization of the expressed protein. Arch Biochem Biophys 283(2):311-317.
37. De Flora A, Zocchi E, Guida L, Franco L, & Bruzzone S (2004) Autocrine and paracrine calcium signaling by the CD38/NAD+/cyclic ADP-ribose system. Ann NY Acad Sci 1028:176-191.
38. Zocchi E, et al. (1998) Expression of CD38 increases intracellular calcium concentration and reduces doubling time in HeLa and 3T3 cells. J Biol Chem 273(14):8017-8024.
39. Zocchi E, et al. (1999) Ligand-induced internalization of CD38 results in intracellular Ca2+ mobilization: role of NAD+ transport across cell membranes. FASEB J 13(2):273-283.
40. Franco L, et al. (1998) The transmembrane glycoprotein CD38 is a catalytically active transporter responsible for generation and influx of the second messenger cyclic ADP-ribose across membranes. FASEB J 12(14):1507-1520.
41. Bruzzone S, Guida L, Zocchi E, Franco L, & De Flora A (2001) Connexin 43 hemi channels mediate Ca2+-regulated transmembrane NAD+ fluxes in intact cells. FASEB J 15(1):10-12.
42. Bruzzone S, et al. (2001) A self-restricted CD38-connexin 43 cross-talk affects NAD+ and cyclic ADP-ribose metabolism and regulates intracellular calcium in 3T3 fibroblasts. J Biol Chem 276(51):48300-48308.
43. Barnes C J, et al. (2003) Functional inactivation of a transcriptional corepressor by a signaling kinase. Nature structural biology 10(8):622-628.
44. Kremer W, Steiner G, Beraud-Dufour S, & Kalbitzer H R (2004) Conformational states of the small G protein Arf-1 in complex with the guanine nucleotide exchange factor ARNO-Sec7. J Biol Chem 279(17):17004-17012.
45. van der Veer M S, et al. (2011) The therapeutic human CD38 antibody daratumumab improves the anti-myeloma effect of newly emerging multi-drug therapies. Blood cancer journal 1(10):e41.
46. Hersher R (2012) Companies wager high on CD38-targeting drugs for blood cancer. Nature medicine 18(10):1446.

1. A method for identifying a molecule acting as an anti-tumoral and/or an anti-proliferative and/or an inhibitor of the fission machinery involved in mitotic Golgi partitioning and/or a modulator of C-terminal-binding proteins (CtBPs) corepressor activity, comprising the steps of:
assaying candidate molecules for their affinity binding for the Rossmann fold of C-terminal-binding proteins (CtBPs);
selecting molecules having an high affinity binding for the Rossmann fold of C-terminal-binding proteins (CtBPs);
testing such high affinity binding molecules for their capacity of inhibiting proliferation and/or inducing an apoptotic response in a cell system.

2. The method according to claim 1 wherein the candidate molecules are previously selected from a database through virtual docking on the C-terminal-binding proteins (CtBPs).

3. The method according to claim 1 wherein the Rossmann fold belongs to the CtBP1-S/BARS (BARS) protein (BARS GenBank accession No. AF067795.

4. The method according to claim 1 wherein the molecule is anti-tumoral against a solid tumor.

5. The method according to claim 1 wherein the cell system used for testing expresses high levels of CD38.

6. A method of treatment of a tumor, comprising administering to a subject in need thereof an effective amount of a molecule selected from the group consisting of:
a) a BFA-ADPR conjugate, said conjugate being formed by ADP-ribosyl cyclase activity, or
b) an inhibitor of binding to BARS of the BFA-ADPR conjugate and/or of NAD/NADH and/or of Acyl CoAs, with the proviso that said inhibitor is not gossypol.

7. The method according to claim 6 wherein the tumor is characterized by high levels of CD38 expression.

8. The method according to claim 6, wherein the inhibitor is selected from the group consisting of:
the compound of formula (I): 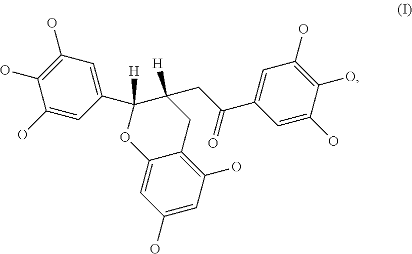
the compound of formula (II): 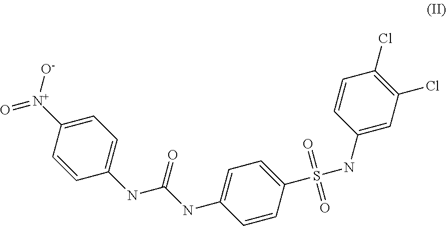 [SMILES: O=C(Nc1ccc([N+](=O)[O-])cc1)Nc1ccc(S(=O)(=O)Nc2ccc(Cl)c(Cl)c2)cc1]
dicumarol, coumermycin A1, salts and derivatives thereof.

9. A method of treating a tumor, comprising administering to a subject in need thereof an effective amount of a molecule able to selectively and with high affinity bind to the Rossmann fold of C-terminal-binding proteins (CtBPs) selected from the group consisting of:
a) a BFA-ADPR conjugate, said conjugate being formed by ADP-ribosyl cyclase activity, or
b) an inhibitor of binding to BARS of the BFA-ADPR conjugate and/or of NAD/NADH and/or of Acyl CoAs, with the proviso that said inhibitor is not gossypol.

10. The method according to claim 9 wherein the tumor is characterized by high levels of CD38 expression.

11. The method according to claim 9 wherein the inhibitor is selected from the group consisting of:
the compound of formula (I): 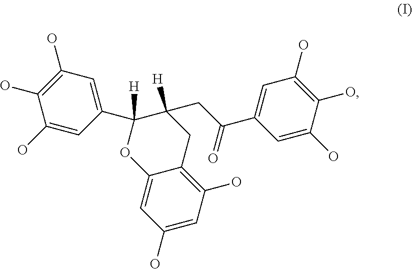
the compound of formula (II): 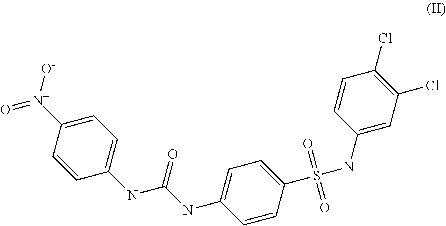 [SMILES: O=C(Nc1ccc([N+](=O)[O-])cc1)Nc1ccc(S(=O)(=O)Nc2ccc(Cl)c(Cl)c2)cc1]
dicumarol, coumermycin A1 salts and derivatives thereof.

12. The method according to claim 6, wherein the tumor is a solid tumor.

13. The method of claim 2, wherein the C-terminal-binding proteins (CtBPs) is the CtBP1-S/BARS (BARS) protein (BARS GenBank accession No. AF067795).

14. The method according to claim 4, wherein the solid tumor is selected from the group consisting of breast, colon, lung cancer and melanoma.

15. The method according to claim 12, wherein the solid tumor is selected from the group consisting of breast, colon, lung cancer and melanoma.


SEQUENCE LISTING (22 sequences)

SEQ ID NO: 1 - protein, 6 aa, Homo sapiens
  MISC_FEATURE: X is any of the 20 natural amino acids
GXGXXG

SEQ ID NO: 2 - DNA, 20 bp, Artificial Sequence
  synthetic primer
tgctgacctgctggattaca

SEQ ID NO: 3 - DNA, 20 bp, Artificial Sequence
  synthetic primer
cctgaccaaggaaagcaaag

SEQ ID NO: 4 - DNA, 20 bp, Artificial Sequence
  synthetic primer
ggggacgaactggacagtaa

SEQ ID NO: 5 - DNA, 20 bp, Artificial Sequence
  synthetic primer
ctgtaatcccagctccttgg

SEQ ID NO: 6 - DNA, 20 bp, Artificial Sequence
  synthetic primer
gacaccactggagggtgact

SEQ ID NO: 7 - DNA, 20 bp, Artificial Sequence
  synthetic primer
ggcgtttggagtggtagaaa

SEQ ID NO: 8 - DNA, 22 bp, Artificial Sequence
  synthetic primer
tcctatggctctgcaattgtca

SEQ ID NO: 9 - DNA, 21 bp, Artificial Sequence
  synthetic primer
ggcaggagtgaatggctcttc

SEQ ID NO: 10 - DNA, 22 bp, Artificial Sequence
  synthetic primer
caacatacagtgacgcttcaca

SEQ ID NO: 11 - DNA, 22 bp, Artificial Sequence
  synthetic primer
cactattgacgtttccccactc

SEQ ID NO: 12 - DNA, 20 bp, Artificial Sequence
  synthetic primer
ctgtggccactacactcagg

SEQ ID NO: 13 - DNA, 20 bp, Artificial Sequence
  synthetic primer
agagcgtcaaagccagaaac

SEQ ID NO: 14 - DNA, 21 bp, Artificial Sequence
  synthetic primer
ggtgggtggtgagatcaatgt

SEQ ID NO: 15 - DNA, 20 bp, Artificial Sequence
  synthetic primer
cgcggttcagttcctctgtc

SEQ ID NO: 16 - DNA, 21 bp, Artificial Sequence
  synthetic primer
atcaccatcttccaggagcga

SEQ ID NO: 17 - DNA, 20 bp, Artificial Sequence
  synthetic primer
gccagtgagcttcccgttca

SEQ ID NO: 18 - DNA, 21 bp, Artificial Sequence
  synthetic primer
tccaagtgccgaaaaaggaag

SEQ ID NO: 19 - DNA, 21 bp, Artificial Sequence
  synthetic primer
cgagttctgagctttcaaggt

SEQ ID NO: 20 - DNA, 23 bp, Artificial Sequence
  synthetic primer
tgaaggtgacagagcctctggat

SEQ ID NO: 21 - DNA, 20 bp, Artificial Sequence
  synthetic primer
tgggtgaattcgggcttgtt

SEQ ID NO: 22 - protein, 420 aa, Homo sapiens
MNGPLHPRPLVALLDGRDCTVEMPILKDLATVAFCDAQSTQEIHEKVLNEAVGAMMYHTITLTREDLEKFKALRVIVRIGSGYDNVDIKAAGELGIAVCNIPSAAVEETADSTICHILNLYRRNTWLYQALREGTRVQSVEQIREVASGAARIRGETLGLIGFGRTGQAVAVRAKAFGFSVIFYDPYLQDGIERSLGVQRVYTLQDLLYQSDCVSLHCNLNEHNHHLINDFTIKQMRQGAFLVNAARGGLVDEKALAQALKEGRIRGAALDVHESEPFSFAQGPLKDAPNLICTPHTAWYSEQASLEMREAAATEIRRAITGRIPESLRNCVNKEFFVTSAPWSVIDQQAIHPELNGATYRYPPGIVGVAPGGLPAAMEGIIPGGIPVTHNLPTVAHPSQAPSPNQPTKHGDNREHPNEQ